SEQUENCE LISTING (39 sequences)

SEQ ID NO: 1 - protein, 159 aa, synthetic construct
AATTVNGGTVHFKGEVVNAACAVDAGSVDQTVQLGQVRTASLAQEGATSSAVGFNIQLNDCDTNVASKAAVAFLGTAIDAGHTNVLALQSSAAGSATNVGVQILDRTGAALTLDGATFSSETTLNNGTNTIPFQARYFATGAATPGAANADATFKVQYQ

SEQ ID NO: 2 - protein, 182 aa, Escherichia coli
MKIKTLAIVVLSALSLSSTAALAAATTVNGGTVHFKGEVVNAACAVDAGSVDQTVQLGQVRTASLAQEGATSSAVGFNIQLNDCDTNVASKAAVAFLGTAIDAGHTNVLALQSSAAGSATNVGVQILDRTGAALTLDGATFSSETTLNNGTNTIPFQARYFATGAATPGAANADATFKVQYQ

SEQ ID NO: 3 - DNA, 546 bp, synthetic construct
  source 1..546: mol_type = other DNA
aaaattaaaactctggcaatcgttgttctgtcggctctgtccctcagttctacagcggctctggccgctgccacgacggttaatggtgggaccgttcactttaaaggggaagttgttaacgccgcttgcgcagttgatgcaggctctgttgatcaaaccgttcagttaggacaggttcgtaccgcatcgctggcacaggaaggagcaaccagttctgctgtcggttttaacattcagctgaatgattgcgataccaatgttgcatctaaagccgctgttgcctttttaggtacggcgattgatgcgggtcataccaacgttctggctctgcagagttcagctgcgggtagcgcaacaaacgttggtgtgcagatcctggacagaacgggtgctgcgctgacgctggatggtgcgacatttagttcagaaacaaccctgaataacggaaccaataccattccgttccaggcgcgttattttgcaaccggggccgcaaccccgggtgctgctaatgcggatgcgaccttcaaggttcagtatcaataa

SEQ ID NO: 4 - DNA, 90 bp, synthetic construct
  source 1..90: mol_type = other DNA
  modified_base 1: mod_base = OTHER; note = Phosphorothioate Bond
  modified_base 2: mod_base = OTHER; note = Phosphorothioate Bond
attcagggttgtttctgaactaaatgtcgcaccatccagcgtcagcgcgaaacccgttctgtccaggatctgcacaccaacgtttgttgc

SEQ ID NO: 5 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacgttggtatgaccgaaatcaatcgccgtacctaaaaaggcaacagcggctttagatgcaacattggt

SEQ ID NO: 6 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacgttggtgaaacccgcatcaatcgccgtacctaaaaaggcaacagcggctttagatgcaacattggt

SEQ ID NO: 7 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
attcagggttgtttctgaactaaatgtcgcaccatccagcgtcagcgcataacccgttctgtccaggatctgcacaccaacgtttgttgc

SEQ ID NO: 8 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacgttggtatgaccataatcaatcgccgtacctaaaaaggcaacagcggctttagatgcaacattggt

SEQ ID NO: 9 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacgttggtataacccgcatcaatcgccgtacctaaaaaggcaacagcggctttagatgcaacattggt

SEQ ID NO: 10 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacgttggtgaaaccgaaatcaatcgccgtacctaaaaaggcaacagcggctttagatgcaacattggt

SEQ ID NO: 11 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacgttggtataaccataatcaatcgccgtacctaaaaaggcaacagcggctttagatgcaacattggt

SEQ ID NO: 12 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacgttggtgaaaccataatcaatcgccgtacctaaaaaggcaacagcggctttagatgcaacattggt

SEQ ID NO: 13 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacgttggtataaccgaaatcaatcgccgtacctaaaaaggcaacagcggctttagatgcaacattggt

SEQ ID NO: 14 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
attcagggttgtttctgaactaaatgtcgcaccatccagcgtcagcgcccaacccgttctgtccaggatctgcacaccaacgtttgttgc

SEQ ID NO: 15 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
acccgcagctgaactctgcagagccagaacgttggtatgaccccaatcaatcgccgtacctaaaaaggcaacagcggctttagatgcaac

SEQ ID NO: 16 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
gcgctacccgcagctgaactctgcagagccagaacgaaggtatgaccgaaatcaatcgccgtacctaaaaaggcaacagcggctttagat

SEQ ID NO: 17 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
agctgaactctgcagagccagaacataggtatgaccataatcaatcgccgtacctaaaaaggcaacagcggctttagatgcaacattggt

SEQ ID NO: 18 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
gcgctacccgcagctgaactctgcagagccagaacgaaggtatgacccgcatcaatcgccgtacctaaaaaggcaacagcggctttagat

SEQ ID NO: 19 - DNA, 90 bp, synthetic construct
  source 1..90: mol_type = other DNA
  modified_base 1: mod_base = OTHER; note = phosphorothioate bond
  modified_base 2: mod_base = OTHER; note = phosphorothioate bond
gcgctacccgcagctgaactctgcagagccagaacataggtatgacccgcatcaatcgccgtacctaaaaaggcaacagcggctttagat

SEQ ID NO: 20 - DNA, 23 bp, synthetic construct
  source 1..23: mol_type = other DNA
gggtcataccaacgttctggctc

SEQ ID NO: 21 - DNA, 24 bp, synthetic construct
  source 1..24: mol_type = other DNA
taatcaatcgccgtacctaaaaag

SEQ ID NO: 22 - DNA, 22 bp, synthetic construct
  source 1..22: mol_type = other DNA
ggcgctgacgctggatggtgcg

SEQ ID NO: 23 - DNA, 24 bp, synthetic construct
  source 1..24: mol_type = other DNA
taacccgttctgtccaggatctgc

SEQ ID NO: 24 - protein, 306 aa, Methanocaldococcus jannaschii
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 25 - DNA, 77 bp, synthetic construct
  source 1..77: mol_type = other DNA
ccggcggtagttcagcagggcagaacggcggactctaaatccgcatggcaggggttcaaatcccctccgccggacca

SEQ ID NO: 26 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 27 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 28 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKCPEKEGGDLTVNSYEELESLFKNKELHPMRLKNAVAEELIKILEPIRKRL

SEQ ID NO: 29 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 30 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKCPEKEGGDLTVNSYEELESLFKNKELHPMRLKNAVAEELIKILEPIRKRL

SEQ ID NO: 31 - protein, 306 aa, synthetic construct
MDEFEMIKRNTSEIISEEELREVLKKDEKSALIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 32 - protein, 306 aa, synthetic construct
MDEFEMIKRNTSEIISEEELREVLKKDEKSALIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 33 - protein, 306 aa, synthetic construct
MDEFEMIKRNTSEIISEEELREVLKKDEKSALIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVDVGGMEQRKIHMLARELLPKKVVCIHNPVLTGLDGEGKMSSSKGNFIAVDDSPEEIRAKIKKAYCPAGVVEGNPIMEIAKYFLEYPLTIKCPEKEGGDLTVNSYEELESLFKNKELHPMRLKNAVAEELIKILEPIRKRL

SEQ ID NO: 34 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNVMHYDGVDVYVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 35 - protein, 307 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNVMHYDGVDVYVGGMEQRKIHMLARELLPKKVVCIHNPVLTGLDGEGKMSSSKGNFIAVDDSPEEIRAKIKKAYCPAGVVEGNPIMMEIAKYFLEYPLTIKGPEKFGGDLTVNSYEELESLFKNKELHPMRLKNAVAEELIKILEPIRKRL

SEQ ID NO: 36 - protein, 307 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNVMHYDGVDVYVGGMEQRKIHMLARELLPKKVVCIHNPVLTGLDGEGKMSSSKGNFIAVDDSPEEIRAKIKKAYCPAGVVEGNPIMMEIAKYFLEYPLTIKCPEKEGGDLTVNSYEELESLFKNKELHPMRLKNAVAEELIKILEPIRKRL

SEQ ID NO: 37 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSTYMLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 38 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSTYMLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 39 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSTYMLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKCPEKEGGDLTVNSYEELESLFKNKELHPMRLKNAVAEELIKILEPIRKRL

(19) United States
(12) Patent Application Publication
(10) Pub. No.: US 2025/0230198 A1
(43) Pub. Date: Jul. 17, 2025

(54) COMPOSITIONS AND METHODS FOR MAKING AND USING PROTEIN NANOWIRES WITH TUNABLE FUNCTIONALITY

(71) Applicant: Yale University, New Haven, CT (US)

(72) Inventors: Farren Isaacs, Stamford, CT (US); Nikhil Malvankar, New Haven, CT (US); Daniel Mark Shapiro, Durham, NC (US); Sibel Ebru Yalcin, Milford, CT (US); Gunasheil Mandava, Sewickley, PA (US)

(21) Appl. No.: 18/833,335

(22) PCT Filed: Jan. 25, 2023
(86) PCT No.: PCT/US2023/061278

§ 371 Date: Jul. 25, 2024

Related U.S. Application Data
(60) Provisional application No. 63/302,932, filed on Jan. 25, 2022.

(51) Int. Cl. C07K 14/245; C12N 1/20; C12N 15/70; C12R 1/19; G01N 27/327

(52) U.S. Cl. CPC C07K 14/245; C12N 1/20; C12N 15/70; G01N 27/3278; C12R 2001/19

(57) ABSTRACT

Engineered, electrically conductive fimbrial polypeptides, pilus, and bundled pili are provided. The polypeptides typically include one or more mutations (e.g., substitution or addition) with an aromatic amino acid relative to the corresponding wildtype fimbrial protein. In some embodiments, the amino acid is a substrate for a “click” chemistry reaction such as Copper(I)-catalyzed azide-alkyne cycloaddition (CuAAC), which can be used to conjugate a functional moiety to the non-standard aromatic amino acid residues. Preferred functional moieties include conductive materials such as metal (e.g., gold) particles, optionally nanoparticles, and heme groups. Also provided are pilus formed of a plurality of the engineered fimbrial polypeptides, and bundles of pili formed of a plurality of the pili. Electrical circuits, devices, and systems including the engineered materials, wherein the engineered material serves as the conductive element, and method of use thereof are also provided. Exemplary devices include, but are not limited to, sensors, transistors, and capacitors.

### CROSS-REFERENCE TO RELATED APPLICATIONS

This application claims the benefit and priority to U.S. Ser. No. 63/302,932 filed Jan. 25, 2022, and which is incorporated by reference herein in its entirety.

### STATEMENT REGARDING FEDERALLY SPONSORED RESEARCH OR DEVELOPMENT

This invention was made with government support under GM140481 and AI138259 awarded by National Institutes of Health and under 1714860 and 1749662 awarded by the National Science Foundation. The government has certain rights in the invention.

### REFERENCE TO THE SEQUENCE LISTING

The Sequence Listing submitted as a text file named “YU_8319_PCT_ST26.xml” created on Jan. 24, 2023, and having a size of 59,140 bytes is hereby incorporated by reference pursuant to 37 C.F.R. § 1.834(c)(1).

### FIELD OF THE INVENTION

The field of the invention is generally related to conductive organic and hybrid organic-inorganic nanowires.

### BACKGROUND OF THE INVENTION

Materials produced from synthetic chemical processes provide access to a broad range of chemical structures yet are constrained by the lack of sequence-defined polymerization methods. In contrast, biological systems employ sequence-controlled processes to synthesize biomolecules, in which the molecular information encoded by nucleic acids is converted into sequence-controlled protein polymers (Lutz et al., Science 341, 1238149 (2013)). Protein polymers have evolved to assume specialized functions in nature, among which is the formation of dynamic protein-based materials (e.g., collagen, silk, and elastin). These multifunctional materials possess versatile functions spanning a range of strength, elasticity, and stability, but lack electronic or optical functionality. Engineered living materials with programmable functionalities and environmental resilience are attractive biomaterials due to their ability to regenerate, sense, and adapt to environmental cues (Gonzalez et al., Nature Chemical Biology 16, 126-133 (2020)). However, nature is constrained to a small set of organic monomeric building blocks, the 20 canonical amino acids, thereby limiting the chemical diversity of polymeric biomaterials. Expanding the chemical palette of genetically encoded chemistries could yield new classes of enzymes, materials, and therapeutics produced in a sequence-defined manner with diverse chemistries.

Many bacteria produce filamentous protein appendages on their surface called pili, which are critical to bacterial infections due to their roles in host colonization and surface sensing (Lillington et al., Biochimica et Biophysica Acta (BBA)—General Subjects 1840, 2783-2793 (2014)), bacterial motility (Burrows, Annu Rev Microbiol 66, 493-520 (2012)), and natural competence (Adams et al., Nat. Microbiol. 4, 1545-1557 (2019)). In addition to their biomedical importance, pili filaments are attractive biomaterials due to their capacity to self-assemble through natural polymerization while retaining extraordinary mechanical stability and robustness, being able to withstand a wide range of temperatures, pH, and protein-denaturing agents such as SDS and urea (Li et al., Journal of Molecular Biology 418, 47-64 (2012), Alonso-Caballero et al., Nature Communications 9, 2758 (2018), Echelman et al., Proceedings of the National Academy of Sciences 113, 2490-2495 (2016), Hospenthal et al., Structure 25, 1829-1838.e1824 (2017)). However, there are several major challenges in the use of pili as multifunctional biomaterials. First, like most proteins, pili lack electronic or optical functionality, which are important for the development of next-generation bioelectronics. It was previously thought that some soil bacteria such as Geobacter sulfurreducens produce conductive type IV pili. However, structural studies revealed that conductive filaments on the bacterial surface are polymerized cytochromes whereas pili remain inside the cell and are involved in the secretion of filamentous cytochromes (Gu et al., “Structure of Geobacter pili reveals secretory rather than nanowire behaviour.” Nature (2021), Wang et al., Cell 177, 361-369.e310 (2019), Yalcin et al., Nature Chemical Biology 16, 1136-1142 (2020)). G. sulfurreducens “pili”, comprised of the PilA-N and PilA-C proteins, the type IV pili pilin proteins conserved throughout many bacterial species (Craig et al., Nature Reviews Microbiology 17, 429-440 (2019)), extend past the bacterial surface only when overexpressed and show very low conductivity (Gu et al., Nature (2021)).

Some reports have claimed conductivity in synthetic “pili” (Ueki et al., ACS Synthetic Biology 9, 647-654 (2020)), however the conductivity of the individual synthetic filaments has not been demonstrated along their length, only across their diameter, providing no evidence that the pili could be conductive down their length like a nanowire. Furthermore, their exact biochemical composition is unknown as discussed at length in a previous study (Gu et al., Nature (2021)). Second, structures of these putative conductive “pili” are not available, hindering the elucidation and prediction of structure-function correlations.

Another obstacle in using pili as multifunctional biomaterials is that, although in vitro assembly of conductive proteins is feasible, they tend to aggregate, which is not suitable for mass production (Ueki et al., ACS Synthetic Biology 9, 647-654 (2020)). Thus, there remains a need a need for improved tools and techniques in this area.

Therefore, it is an object of the invention to provide compositions and methods for in vivo biomaterial production.

It is a further object of the invention to provide biomaterials endowed with tunable electronic and mechanical functionalities, compositions or making and using the same.

### SUMMARY OF THE INVENTION

Engineered, electrically conductive fimbrial polypeptides are provided. The polypeptides typically include one or more mutations (e.g., substitution or addition), typically with an aromatic amino acid, relative to the corresponding wildtype fimbrial protein. The aromatic amino acid can be a canonical amino acid such as phenylalanine, tyrosine, histidine, or tryptophan; or a non-standard amino acid such as propargyloxy-phenylalanine (PrOF), p-azido-1-phenylalanine (pAzF), 3-(2-Naphthyl)-L-alanine (2NaA), or others mentioned below and elsewhere. In some embodiments, the amino acid is a substrate for a “click” chemistry reaction such as Copper(I)-catalyzed azide-alkyne cycloaddition (CuAAC), which can be used to conjugate a functional moiety to the non-standard aromatic amino acid residues. Preferred functional moieties include metal (e.g., gold) particles, optionally nanoparticles, and heme groups. In some embodiments, the functional moiety increases or enhances the conductivity of the fimbrial polypeptide or pili formed therefrom.

In some embodiments, the wildtype fimbrial protein is FimA, optionally E. coli FimA or an ortholog, paralog, or homolog thereof. In some embodiments, the FimA includes the amino acid sequence of SEQ ID NO:1. In some embodiments, the engineered polypeptide includes two or more mutations optionally<15 Å apart in pilus formed thereof. Mutations can be presented with reference to SEQ ID NO:1 or the corresponding amino acids in other wildtype fimbrial proteins. For example, in some embodiments, the engineered polypeptide includes mutations at one or more of A80, H82, and A109 relative to the wildtype protein. Exemplary, non-limiting specific mutations are A80F, A109F, A80Y, A80 W, A109 W, A80F A109F (double mutant), A109Y, A80Y A109Y (double mutant), A80 W A109 W (double mutant), A109/PrOF, A80/2NaA, and A80/pAzF.

Also provided are pili formed of a plurality of the engineered fimbrial polypeptides, and bundles of pili formed of a plurality of the pili. The bundles can be unordered or ordered and form a 1D, 2D, or 3D structure. A non-limiting exemplary structure is a lattice structure. In some embodiments, the structures are form by self-assembled following contract with an inducer, such as hexamethylenediamine (HMD), pimelic acid, or 1,3-propanedisulfonic acid.

Preferably, the fimbrial polypeptide, pilus, or bundle of pili is more conductive than the corresponding wildtype fimbrial polypeptide, pilus, or bundle of pili. Electrical circuits, devices, and systems including the fimbrial polypeptide, pilus, or bundle of pili, wherein the fimbrial polypeptide, pilus, or bundle of pili serves as the conductive element, and methods of use thereof are also provided. Exemplary devices include, but are not limited to, sensors, transistors, and capacitors.

Methods of making the fimbrial polypeptide, pili, and bundles of pili are also provided. For example, a methods of making a fimbrial polypeptide having one or more iterations of an non-standard amino acid, e.g., aromatic non-standard amino acid, can include expressing a messenger RNA (mRNA) encoding the fimbrial polypeptide in a system including an orthogonal translation system (OTS) including a nucleic acid sequence encoding an aminoacyl tRNA synthetase (AARS) and its cognate tRNA operably linked to expression control sequences and transformed, transfected, or integrated into a genomically recoded organism (GRO) with at least one codon reduced or absent from its genome, and a plurality of the non-standard amino acid. Typically, the mRNA includes a nucleic acid sequence having at least one iteration of the codon deleted from the GRO, the AARS can charge the tRNA with non-standard amino acid, the tRNA includes an anticodon that can bind to the codon reduced or absent from the GRO.

A method of making pili can including making the fimbrial polypeptide in a prokaryotic host, optionally wherein the prokaryotic host is E. coli, and isolating pili formed by the host.

A method of forming an ordered bundle of pili can include isolating pili, and contacting the pili with an inducer, optionally wherein the inducer is HMD.

### BRIEF DESCRIPTION OF THE DRAWINGS

FIGS. 1A-1D illustrate a strategy to engineer electronic conductivity into E. coli pili nanofilaments. FIG. 1A is a representative TEM image of an E. coli cell (left) expressing pili and purified pili (right). Scale bars, 200 nm (left) and 100 nm (right). FIG. 1B is a cryo-EM structure of 8 FimA monomers forming mature pilus. Positions 80 and 109 in each monomer are highlighted with different colored spheres: side view (left), front view (right). FIGS. 1A-1B, experiments replicated independently greater than 15 times with similar results. FIG. 1C is an illustration showing a strategy to develop hierarchical ordered structures with enhanced conductivity. FIG. 1D is a schematic of creating organic-inorganic hybrid pili using gold nanoparticles clicked on through azido-alkyne click chemistry functionality encoded with nsAAs.

FIGS. 2A-2E illustrate electronic conductivity of individual pili. FIG. 2A is a schematic of measurements and AFM image of pili bridging the gold electrodes. Scale bar, 200 nm. FIG. 2B is a graph showing the height profile of pili at location (black bar crossing pilus) shown in FIG. 2A. FIGS. 2C-2D are graphs showing current-voltage profile of pili with different aromatic residue mutations, each line representative of conductivity measurements on one pilus. Representative points were shifted by a constant value such that the slope of the current-voltage curve retained the same value but intercepted at zero for comparison purposes. Currents were measured after applying voltages from −0.15 to 0.15 V in intervals of 0.05. FIG. 2E is a bar graph showing conductivity comparison of pili. Error bars represent s.e.m. (n=3).

FIGS. 3A-3F show computationally-guided design of hierarchical nanostructures. FIG. 3A is an illustration of a strategy to align pili using HMD molecule. FIG. 3B is a plot showing the time evolution of the distance between the geometric centers of each monomer. FIG. 3C is a histogram displaying the distribution of distances between the geometric centers of the pilin monomers in the presence and absence of 250 mM HMD. Data was collected from separate 100 ns simulations. FIG. 3D is an illustration of the time evolution of the interaction between two FimA monomers in presence and absence of 250 mM HMD. FIG. 3E is AFM images of pili on mica (Scale bar, 200 nm) and a plot of the height profile (right) of pili at location shown (black bar crossing bundle) in middle image confirms the bundling. FIG. 3F is a plot showing conductivity comparison of ordered pili. Error bars represent s.e.m. (n=3).

FIGS. 4A-4E show hybrid organic-inorganic nanowires with ˜170-fold higher conductivity through site-specific incorporation of nsAAs conjugated to gold nanoparticles (AuNPs). FIG. 4A is an AFM image of AuNP-pili resulting from reacting azide-functionalized AuNPs with PrOF-containing pili with copper added to the Cu-catalyzed click chemistry reaction. Scale bar 100 nm, and a graph showing the corresponding height profile (below). Experiment independently repeated three times with similar results simultaneously with experiments performed for FIG. 4B. FIG. 4B is an AFM image of naked PrOF-containing pili resulting from reacting azide-functionalized AuNPs with PrOF-containing pili without copper added to the Cu-catalyzed click chemistry reaction. Scale bar 20 nm, and a graph showing the corresponding height profile (below). Experiment independently repeated three times with similar results simultaneously with experiments performed for FIG. 4A. FIGS. 4C and 4D are plots showing current-voltage profile of pili incorporating 2NaA (4C) and PrOF conjugated with AuNP (4D). Representative points were shifted by a constant value such that the slope of the current-voltage curve retained the same value but intercepted at zero for comparison purposes. FIG. 4E is a bar graph showing conductivity of pili biomaterials incorporating nsAAs. Error bars represent s.e.m (n=3).

FIGS. 5A-5E illustrate AuNP-decorated Pili. FIG. 5A-5C are AFM images of AuNP-decorated pili demonstrate consistent coverage of Type 1 pili with incorporated PrOF with azide-functionalized AuNPs after Cu-catalyzed click reaction. FIG. 5A is a representative AuNP-decorated pili. FIG. 5B is a zoomed in view of the box in 5A. FIG. 5C is a zoomed in (single) AuNP-decorated pilus of the box in 5B, and graph illustrating the associated height profiles. Pili imaged after Cu-catalyzed click reaction are consistently 2 nm in diameter, purchased AuNPs are 5 nm in diameter. Cross-section of AuNPs attached to pilus is 7 nm, indicating AuNP with pilus protein underneath. For parts 5A-5C, experiments were repeated independently 3 times with similar results. FIG. 5D is an illustration of 5 nm N Hydroxysuccinimide (NHS)-functionalized AuNPs. To change the terminal NHS group to an azide group, the NHS group was covalently bound to the amine group of the 11-Azido-3,6,9-trioxaundecan-1-amine azide linker (methods). FIG. 5E is representative AFM images of AuNP-decorated pilus crossing two electrodes, and graph showing the height profile corresponds to height across diameter of pilus+AuNPs. As seen from this image, in all measured cases AuNPs decorated entire pilus between electrodes.

### DETAILED DESCRIPTION OF THE INVENTION

### I. Definitions

As used herein, the terms “transfer RNA” and “tRNA” refers to a set of genetically encoded RNAs that act during protein synthesis as adaptor molecules, matching individual amino acids to their corresponding codon on a messenger RNA (mRNA). tRNAs assume a secondary structure with four base paired stems known as the cloverleaf structure. The tRNA contains a stem and an anticodon. The anticodon is complementary to the codon specifying the tRNA's corresponding amino acid. The anticodon is in the loop that is opposite of the stem containing the terminal nucleotides. The 3′ end of a tRNA is aminoacylated by a tRNA synthetase so that an amino acid is attached to the 3′end of the tRNA. This amino acid is delivered to a growing polypeptide chain as the anticodon sequence of the tRNA reads a codon triplet in an mRNA.

As used herein, the term “anticodon” refers to a unit made up of typically three nucleotides that correspond to the three bases of a codon on the mRNA. Each tRNA contains a specific anticodon triplet sequence that can base-pair to one or more codons for an amino acid or a “stop codon.” “Stop codons” can act as a signal for termination of protein synthesis (i.e., do not codon for an amino acid), or can be repurposed to encode amino acids (including non-standard amino acids) by engineered translation machinery. Known “stop codons” include, but are not limited to, the three codon bases, UAA known as ochre, UAG known as amber and UGA known as opal. tRNAs do not decode stop codons naturally, but can and have been engineered to do so. Stop codons are usually recognized by enzymes (release factors) that cleave the polypeptide as opposed to encode an amino acid (AA) via a tRNA.

As used herein, the term “suppressor tRNA” refers to a tRNA that alters the reading of a messenger RNA (mRNA) in a given translation system. For example, a nonsense suppressor tRNA can read through a stop codon.

As used herein, the term “aminoacyl tRNA synthetase (AARS)” refers to an enzyme that catalyzes the esterification of a specific amino acid or its precursor to one of all its compatible cognate tRNAs to form an aminoacyl-tRNA. These charged aminoacyl tRNAs then participate in mRNA translation and protein synthesis. The AARS show high specificity for charging a specific tRNA with the appropriate amino acid. In general, there is at least one AARS for each of the twenty amino acids.

As used herein, the term “residue” refers to an amino acid that is incorporated into a protein. The amino acid may be a naturally occurring amino acid and, unless otherwise limited, may encompass known analogs of natural amino acids that can function in a similar manner as naturally occurring amino acids.

As used herein, the terms “polynucleotide” and “nucleic acid sequence” refers to a natural or synthetic molecule including two or more nucleotides linked by a phosphate group at the 3′ position of one nucleotide to the 5′ end of another nucleotide. The polynucleotide is not limited by length, and the polynucleotide can include deoxyribonucleic acid (DNA) or ribonucleic acid (RNA).

As used herein, the term “conservative variant” refers to a particular nucleic acid sequence that encodes identical or essentially identical amino acid sequences. Conservative substitution tables providing functionally similar amino acids are well known in the art. The following sets forth exemplary groups which contain natural amino acids that are “conservative substitutions” for one another. Conservative Substitution Groups 1: Alanine (A), Serine (S), Threonine (T); 2: Aspartic acid (D), Glutamic acid (E); 3: Asparagine (N), Glutamine (Q); 4: Arginine (R), Lysine (K); 5: Isoleucine (I), Leucine (L), Methionine (M) Valine (V); and 6: Phenylalanine (F), Tyrosine (Y), Tryptophan (W).

As used herein, the term “percent (%) sequence identity” or “homology” refers to the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

As used herein, the term “transgenic organism” refers to any organism, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. Suitable transgenic organisms include, but are not limited to, bacteria, cyanobacteria, fungi, plants and animals. The nucleic acids described herein can be introduced into the host by methods known in the art, for example infection, transfection, transformation or transconjugation.

As used herein, the term “eukaryote” or “eukaryotic” refers to organisms or cells or tissues derived from these organisms belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

As used herein, the term “prokaryote” or “prokaryotic” refers to organisms including, but not limited to, organisms of the Eubacteria phylogenetic domain, such as Escherichia coli, Thermus thermophilus, and Bacillus stearothermophilus, or organisms of the Archaea phylogenetic domain such as, Methanocaldococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, and Aeuropyrum pernix.

As used herein, the term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “translation system” refers to the components that facilitate incorporation of an amino acid into a growing polypeptide chain (protein). Key components of a translation system generally include at least AARS and tRNA, and may also include amino acids, ribosomes, AARS, EF-Tu, and mRNA.

As used herein, the term “orthogonal translation system (OTS)” refers to at least an AARS and paired tRNA that are both heterologous to a host or translational system in which they can participate in translation of an mRNA including at least one codon that can hybridize to the anticodon of the tRNA.

As used herein, the terms “recoded organism” and “genomically recoded organism (GRO)” in the context of codons refer to an organism in which the genetic code of the organism has been altered such that a codon has been eliminated from the genetic code by reassignment to a synonymous or nonsynonymous codon.

As used herein, the term “polyspecific” refers to an AARS that can accept and incorporate two or more different non-standard amino acids.

As used herein, the terms “protein,” “polypeptide,” and “peptide” refers to a natural or synthetic molecule comprising two or more amino acids linked by the carboxyl group of one amino acid to the alpha amino group of another. The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus.

As used herein, “standard amino acid” and “canonical amino acid” refer to the twenty amino acids that are encoded directly by the codons of the universal genetic code denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “non-standard amino acid (nsAA)” refers to any and all amino acids that are not a standard amino acid. nsAA can be created by enzymes through posttranslational modifications; or those that are not found in nature and are entirely synthetic (e.g., synthetic amino acids (sAA)). In both classes, the nsAAs can be made synthetically. WO 2015/120287 provides a non-exhaustive list of exemplary non-standard and synthetic amino acids that are known in the art (see, e.g., Table 11 of WO 2015/120287).

As used herein, “genetically modified organism (GMO)” refers to any organism whose genetic material has been modified (e.g., altered, supplemented, etc.) using genetic engineering techniques. The modification can be extrachromosomal (e.g., an episome, plasmid, etc.), by insertion or modification of the organism's genome, or a combination thereof.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product, for example a functional RNA that does not encode a protein or polypeptide (e.g., miRNA, tRNA, etc.). The term gene as used herein with reference to genomic DNA includes intervening, non-coding regions as well as regulatory regions and can include 5′ and 3′untranslated ends. The term gene as used herein with reference to recombinant expression constructs may, but need not, include intervening, non-coding regions, regulatory regions, and/or 5′ and 3′untranslated ends. Thus, with respect to a recombinant expression constructs, a gene may be only an open reading frame (ORF).

As used herein, the term “construct” refers to a recombinant genetic molecule having one or more isolated polynucleotide sequences. Genetic constructs used for transgene expression in a host organism, also referred to “expression constructs”, include in the 5′-3′ direction, a promoter sequence; a sequence encoding a gene of interest; and a termination sequence. The construct may also include selectable marker gene(s) and other regulatory elements for expression.

As used herein, the term “vector” refers to a polynucleotide capable of transporting into a cell another polynucleotide to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector.

As used herein, the term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of gene to a transcriptional control element refers to the physical and functional relationship between the gene and promoter such that the transcription of the gene is initiated from the promoter by an RNA polymerase that specifically recognizes, binds to and transcribes the DNA.

As used herein, term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the term “promoter” refers to a regulatory nucleic acid sequence, typically located upstream (5′) of a gene or protein coding sequence that, in conjunction with various elements, is responsible for regulating the expression of the gene or protein coding sequence. These include constitutive promoters, inducible promoters, tissue- and cell-specific promoters and developmentally-regulated promoters.

As used herein, the terms “transformed,” “transgenic,” “transfected” and “recombinant” refer to a host organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

As used herein, the term “endogenous” with regard to a nucleic acid refers to nucleic acids normally present in the host.

As used here, the term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

Unless otherwise indicated, the disclosure encompasses conventional techniques of molecular biology, microbiology, cell biology and recombinant DNA, which are within the skill of the art. Unless otherwise noted, technical terms are used according to conventional usage, and in the art, such as in the references cited herein, each of which is specifically incorporated by reference herein in its entirety.

### II. Compositions

Bacterial pili serve as an attractive biomaterial for the development of engineered protein materials due to their ability to self-assemble into mechanically robust filaments. However, most biomaterials lack electronic functionality and atomic structures of putative conductive proteins are not known. Disclosed herein are engineered high electronically conductive pili produced by a genomically-recoded E. coli strain. Results presented in the experiments below show that incorporation of tryptophan into pili increased conductivity of individual filaments>80-fold. Furthermore, ordering of the pili into nanostructures increased conductivity 5-fold compared to unordered pili networks. Site-specific conjugation of pili with gold nanoparticles, facilitated by incorporating the nonstandard amino acid propargyloxy-phenylalanine, increased filament conductivity ˜170-fold.

Thus, provided herein are compositions, and methods of making and using sequence-defined, highly-conductive protein nanowires and hybrid organic-inorganic biomaterials with genetically-programmable electronic functionalities not accessible in nature or through chemical-based synthesis.

### A. Fimbrial Polypeptides

Provided herein are fimbrial polypeptides, e.g., engineered FimA polypeptides, modified to enhance their conductivity.

The fimbrial polypeptides are typically a variant of a wildtype fimbrial protein such as fimA, or a functional fragment thereof having one or more mutations adding one or more amino acids, most typically with an aromatic amino acid(s). The mutation(s) can be a substitution, insertion, or a combination thereof. The aromatic amino acids can be canonical (e.g., standard) amino acids, such as phenylalanine, tyrosine, histidine, and tryptophan; non-standard amino acids; or a combination thereof. A preferred standard amino acid is tryptophan. Exemplary non-standard amino acids include, but are not limited to, propargyloxy-phenylalanine (PrOF), p-azido-1-phenylalanine (pAzF), 3-(2-Naphthyl)-L-alanine (2NaA), 4-Chloro-phenylalanine, 4-bromo-phenylalanine, para-acetyl-phenylalanine, para-amino-phenylalanine, 4-Iodo-phenylalanine, phenyl-L-phenylalanine, O-2-azidoethyl-tyrosine, para-azidomethyl-phenylalanine, 4-propargyloxy-1-phenylalanine (pPR), and other mentioned in, e.g., Hadar, et al., Chembiochem, 22(8):1379-1384 (2021). doi: 10.1002/cbic.202000663, Arranz-Gibert, et al., Cell Chem Biol., S2451-9456(21)00516-X. (2021) doi: 10.1016/j.chembiol.2021.12.002, and WO 2015/120287, each of which is specifically incorporated by reference herein in its entirety. In some embodiments, the aromatic non-standard amino acid is or includes one or more of pAcF, pAzF, StyA, 4IF, 4BrF, 4ClF, 4MeF, 4Cf3F, MeY, 4NO2F, 4BuF, BuY, 2NaA, and/or PheF, which can be charged to a cognate tRNA by AARS pAcFRS.2.t1.

In some embodiments, the mutated residue(s) are external residues (e.g., the highly variable region) of the pili formed by the fimbrial polypeptide. In some embodiments, two or more mutant residues are in a set of variable surface residues of an assembled pilus which, when mutated, are in close enough proximity to each other to facilitate efficient electron transfer, likely through electron hopping, along the pilus.

### 1. Exemplary Fimbrial Polypeptide Sequences

Exemplary fimbrial sequences and variant polypeptides are provided. In some embodiments, the wildtype fimbrial protein is a FimA (also referred to herein as Type-1 fimbrial protein, A chain; Type-1A pilin, or by reference to the gene fimA) wildtype protein. FimA wildtype proteins are known in the art. Typically, the FimA wildtype protein is from a prokaryote, most typically a bacterium. In some embodiments, the FimA wildtype is E. coli FimA, or an ortholog, paralog, or homolog thereof, e.g., from another bacterial species.

The sequence of E. coli FimA is known in the art. A consensus sequence is

(SEQ ID NO: 2, UniProt KB-P04128 (FIMA1_ECOLI))
MKIKTLAIVVLSALSLSSTAALAAATTVNGGTVHFKGEVV
NAACAVDAGSVDQTVQLGQVRTASLAQEGATSSAVGFNIQ
LNDCDINVASKAAVAFLGTAID [symbol] G [symbol] TNVLALQSSAAGSAT
NVGVQILDRTGAALTLDGATFSSETTLNNGTNTIPFQARY
FATGAATPGAANADATFKVOYQ,

wherein the signal sequence is represented with italics.

Thus, a consensus E. coli FimA protein sequence without the signal peptide sequence is

(SEQ ID NO: 1)
AATTVNGGTVHFKGEVVNAACAVDAGSVDQTVQLGQVRTA
SLAQEGATSSAVGFNIQLNDCDTNVASKAAVAFLGTAID [symbol]
G [symbol] TNVLALQSSAAGSATNVGVQILDRTGAALTLDGATFSS
ETTLNNGTNTIPFQARYFATGAATPGAANADATFKVQYQ

In some embodiments, the FimA wildtype protein is SEQ ID NO:1 without or with the endogenous or a heterologous signal sequence (e.g., SEQ ID NO:2), or an ortholog, paralog, or homolog thereof, optionally, with at least 50, 60, 70, 75, 80, 85, 90, 95, or more percent sequence identity to SEQ ID NO:1 or 2. In some embodiments, the ortholog, paralog, or homolog thereof is a fimbrial protein, optionally a FimA protein, from another bacterial strain or species. Typically, the ortholog, paralog, or homolog is a fimbrial protein that alone or in combination with another protein(s) forms pili in the organism (e.g., bacteria). In preferred embodiments, the pili is a Type 1 pili.

### 2. Engineered Fimbrial Polypeptides

The engineered fimbrial polypeptides are typically a variant of a wildtype fimbrial protein such as a wildtype FimA protein, or a functional fragment thereof having at least one mutation adding one or more aromatic amino acids. The mutation(s) can be a substitution, insertion, or a combination thereof. In some embodiments, the fimbrial polypeptide has one or more additional mutations that can be substitution(s), insertion(s), deletion(s), or a combination thereof. In some embodiments, the additional mutations are conservative mutations and/or are from a known sequence variant or an ortholog, paralog, or homolog.

Preferably, a plurality of the engineered fimbrial polypeptides maintain the ability to form a pilus and a plurality of the pili can form a pili network. The engineered fimbrial polypeptide monomers, a pilus formed by a plurality the fimbrial polypeptide monomers, and/or a network of the pili preferably has higher electrical conductivity than their corresponding wildtype fimbrial protein monomer, pilus, and/or pili network, respectively.

In some embodiments, the fimbrial polypeptide is a variant of FimA wildtype protein of SEQ ID NO:1 without or with the endogenous or a heterologous signal sequence (e.g., SEQ ID NO:2). Preferred mutations are at locations 80, 82, and/or 109 relative to SEQ ID NO:1 (highlighted in bold dash underline, dotted underline, and solid underline, respectively in SEQ ID NOS:1 and 2, above), or the corresponding positions in a ortholog, paralog, or homolog thereof. Thus, in some embodiments, the fimbrial polypeptide is a variant of FimA wildtype protein of SEQ ID NO:1 without or with the endogenous or a heterologous signal sequence (e.g., SEQ ID NO:2) having one or more aromatic amino acids substituted at 80, 82, and/or 109 relative to SEQ ID NO:1, or the corresponding positions in a ortholog, paralog, or homolog thereof. The substitutions can be with the same or different amino acids. In some embodiments, the polypeptide includes standard and non-standard amino acid substitutions. In other embodiments, all of the substitutions are either standard or non-standard amino acids. Thus, single, double, and higher order mutations with the same or different amino acids are contemplated.

In some embodiments, the substitutions are selected from phenylalanine, tyrosine, histidine, tryptophan, PrOF, p-azido-1-phenylalanine (pAzF), 3-(2-Naphthyl)-L-alanine (2NaA), others mentions above or elsewhere herein or otherwise known in the art, or a combination of any of the foregoing.

Exemplary mutants having standard amino acid substitutions include, but are not limited to, A80F, A109F, A80Y, A80 W, A109 W, A80F A109F (double mutant), A109Y, A80Y A109Y (double mutant), and A80 W A109 W (double mutant). Non-limiting preferred non-standard amino acid substitutions are A109/PrOF, A80/2NaA, and A80/pAzF.

In some embodiments, the engineered fimbrial polypeptide has at least 50, 60, 70, 75, 80, 85, 90, 95, or more percent sequence identity to SEQ ID NO:1 or 2.

### B. Pili and Structured Pili Networks

The engineered fimbrial polypeptides can be used to form a pilus. Type 1 bacterial pilus is a rigid, straight, naturally occurring protein nanorod (6-7 nm wide and ˜300 nm-2 μm long) that can be detached from bacterial cells (Cao, et al., Angew Chem Int Ed Engl. 50(28):6264-8 (2011). doi: 10.1002/anie.201102052.). It is helically assembled of about 1000 or more than 1000 copies of a fimbrial polypeptide, such as FimA, with 27 subunits in eight turns. It has an anionic surface with an isoelectric point of 3.92. Its biological function is to assist the adhesion of bacteria to surfaces, including, but not limited to, solid surfaces or biological host tissues. Thus, such pili can form a structure suitable for use as a long helical nanowire.

The disclosed pili typically have a plurality of the engineered fimbrial polypeptides. Typically, the pilus is formed of a homogenous plurality of a single engineered fimbrial polypeptide. However, combinations of two or more different engineered fimbrial polypeptides, and combinations of wildtype and one or more engineered fimbrial polypeptides are also contemplated.

Pili (e.g., a plurality of engineered pilus) can be used to form pili networks. The pili networks can be organized or unorganized, and can be assembled into 1D, 2D, or 3D structures. A particular, non-limiting network exemplified in the experiments below is an ordered, 2D lattice structure. Pili networks formed of combinations of pilus formed from different fimbrial polypeptides and/or wildtype are also contemplated.

### C. Conjugated Engineered Fimbrial Polypeptides and Pili

In some embodiments, the engineered fimbrial polypeptide includes one or more functional moieties conjugated thereto. The functional moieties are most typically targeted to aromatic amino acid substitution(s) or additions.

Preferably the functional moiety improves the conductivity of the fimbrial polypeptide monomer, pilus including a plurality of fimbrial polypeptide, and/or a pili network formed from a plurality of the pilus, e.g., relative to the corresponding unconjugated form.

Exemplary functional moieties include, but not are not limited to, metal particles including, but not limited to nanoparticles. Preferably the metal is electrically conductive. Examples include, but are not limited to, silver, copper, gold, aluminum, molybdenum, zinc, lithium, brass, nickel, steel, palladium, platinum, tungsten, tin, bronze, carbone, lead, titanium, mercury, and FeCrAl. The metal moiety can be first functionalized with a reactive moiety that is able to be conjugated to the aromatic amino acid through click-chemistry or similar conjugation reaction.

In other embodiments, the functional moiety is a heme group. Heme is composed of a ringlike organic compound known as a porphyrin, to which an metal atom is attached. It is the iron atom that reversibly binds oxygen as the blood travels between the lungs and the tissues. The heme group can be derived from or part of a heme protein such as heme a, heme b, heme c, heme d, heme d1, heme o, etc. For example, the heme functional moieties can be used to enhance conductivity is in G. sulfurreducens nanofilaments (Wang et al., Cell 177, 361-369.e310 (2019), Yalcin et al., Nature Chemical Biology 16, 1136-1142 (2020)). The heme group needs to be first functionalized with a reactive moiety that is able to be conjugated to the amino acid through click-chemistry or similar conjugation reaction.

In other embodiments, photoreactive, cross-linkable nsAAs such as pAzF (Costa et al., Advanced Materials 30, 1704878 (2018)) can be incorporated into pili to generate light-activated arrays of conductive protein biomaterials. Upon irradiation with ultraviolet light, the azide group of the pAzF amino acid forms a reactive nitrene, which reacts with nearby H—N or H—C bonds. See, e.g., Vanderschuren, et al., Proceedings of the National Academy of Sciences. 2022 119(4) e2103099119 (2022) doi: 10.1073/pnas.2103099119, Arranz-Gibert, et al., Cell Chem Biol., S2451-9456(21)00516-X. (2021) doi: 10.1016/j.chembiol.2021.12.002. Epub ahead of print. PMID: 34965380. This reaction cross-links the pAzF residues with nearby pili, thus creating a cross-linked conductive material. In this embodiment, pili are not associated with one another until being irradiated with ultraviolet light—this allows for a “soft” solution of pili before irradiation and a controlled creation of a “hard”, arrested, pili network.

In some embodiments, the functional moieties are attached using “click” chemistry. In chemical synthesis, “click” chemistry is a class of biocompatible small molecule reactions commonly used in bioconjugation, allowing the joining of substrates of choice with specific biomolecules. Examples of “click” chemistry include Copper(I)-catalyzed azide-alkyne cycloaddition (CuAAC), Strain-promoted azide-alkyne cycloaddition (SPAAC), Strain-promoted alkyne-nitrone cycloaddition (SPANC), and strained alkene reactions optionally selected from alkene-azide [3+2]cycloaddition, the alkene-tetrazine inverse-demand Diels-Alder cycloaddition, and the alkene-tetrazole photoclick reaction. As illustrated in the experiments below, organic-inorganic hybrid pili were prepared using azide-functionalized AuNPs gold nanoparticles clicked to PrOF-containing pili using Cu-catalyzed azido-alkyne click chemistry (CuAAC).

The conjugate can be made or added to fimbrial polypeptide monomers or pilus or pili networks formed thereof. Thus, conjugated fimbrial polypeptide monomers, pilus, and pili networks are all expressly disclosed.

### D. Pili-Based Nanowires, Circuits, and Devices

In some embodiments, the engineered fimbrial polypeptide monomers, pili, and/or pili networks, or conjugates thereof are utilized in a larger system or circuit. Typically, the engineered pili or pili networks are utilized as conductive nanowires. In some embodiments, the conductive nanowire is conductive over nanometer or micrometer distances. For example, in some embodiments, the nanowire is conductive over of distance of up to 300 nm, or up to 5 μM. Thus, in some embodiments, the nanowire is conductive for a distance of between 10 nm and 5 μM inclusive, or any specific distance or subrange therebetween.

In some embodiments, one or both ends of fimbrial polypeptide monomers, pili, and/or pili networks, or conjugates thereof (e.g., nanowire) are connect at one or more ends to an electrode (e.g., a power source) and/or a device. Thus, one or more the nanowires can be used to form an electrical circuit. Also provided are electronically active pili in wide range of devices and applications such as sensors, transistors, and capacitors that span biological and/or electrical systems. The disclosed compositions can be substituted for traditional conductive material (e.g., wires) therein. Protein nanowires can also be used in applications spanning the electronic-biological interface e.g., electronic prosthetics, implantable electrodes, flexible electronics, energy storage, soft robotics, computing, and information storage.

The disclosed circuits, devices, and applications including engineered pili can be otherwise utilized in the same manner as those devices utilizing a traditional conductive material, provided the devices are used in a way which does not degrade the engineered pili.

### III. Compositions for Making Engineered Fimbrial Polypeptides and Pili

Compositions and methods for making engineered fimbrial polypeptides are also provided. Any suitable method can be used to form the disclosed fimbrial polypeptides including one or more aromatic amino acid mutations. Several methods of making polypeptides, including nsAA-containing polypeptides, are known in the art. A first approach introduces an nsAA by complete amino acid replacement wherein a natural amino acid is substituted for a close synthetic analog (i.e., the nsAA) in an auxotrophic strain (Dougherty, et al., Macromolecules, 26:1779-1781 (1993)).

Preferably, as discussed in more detail below, nsAAs can be incorporated via codon reassignment or frameshift codons using orthogonal translation systems (OTSs) having an aminoacyl tRNA synthetases (“AARS”) that is only able to charge a cognate tRNA, which is not aminoacylated by endogenous AARSs (Liu, et al., Annu Rev Biochem, 79:413-44 (2010), Chin, et al., Annu Rev Biochem, (2014), Amiram, et al., Nat Biotechnol., 33, 1272-1279 (2015), WO 2015/120287).

### A. Nucleic Acids

Polynucleotides encoding the disclosed proteins and polypeptide, including the engineered fimbrial polypeptides are also disclosed. The polynucleotides can be isolated nucleic acids, incorporated into in a vector, or part of a host genome. The polynucleotides can also be part of a cassette including nucleic acids encoding other translational components such as a paired tRNA, selection marker, promoter and/or enhancer elements, integration sequences (e.g., homology arms), etc.

As used herein, “isolated nucleic acid” refers to a nucleic acid that is separated from other nucleic acid molecules that are present in a genome, including nucleic acids that normally flank one or both sides of the nucleic acid in the genome. The term “isolated” as used herein with respect to nucleic acids also includes the combination with any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule or an RNA molecule, provided one of the nucleic acid sequences normally found immediately flanking that DNA molecule in a naturally-occurring genome is removed or absent. Thus, an isolated nucleic acid includes, without limitation, a DNA molecule or RNA molecule that exists as a separate molecule independent of other sequences (e.g., a chemically synthesized nucleic acid, or a cDNA, or RNA, or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule or RNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids encoding the polypeptides and proteins disclosed herein may be optimized for expression in the expression host of choice. In the case of nucleic acids encoding expressed polypeptides, codons may be substituted with alternative codons encoding the same amino acid to account for differences in codon usage between the organism from which the nucleic acid sequence is derived and the expression host. In this manner, the nucleic acids may be synthesized using expression host-preferred codons.

Nucleic acids can be in sense or antisense orientation, or can be complementary to a reference sequence, for example, a sequence encoding the disclosed polypeptides and protein. Nucleic acids can be DNA, RNA, nucleic acid analogs, or combinations thereof. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

### B. Methods for Producing Isolated Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding the disclosed polypeptides or proteins. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995.

When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also be obtained by mutagenesis. Nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of nucleic acid amino acid positions relative to a reference sequence that can be modified include those described herein.

### C. Constructs and Vectors

Constructs and vectors encoding the disclosed polypeptides and proteins are also provided. Nucleic acids, such as those described above, can be inserted into vectors for expression in cells. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. Operably linked means the disclosed sequences are incorporated into a genetic construct so that expression control sequences effectively control expression of a sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II).

A “promoter” as used herein is a DNA regulatory region capable of initiating transcription of a gene of interest. Some promoters are “constitutive,” and direct transcription in the absence of regulatory influences. Some promoters are “tissue specific,” and initiate transcription exclusively or selectively in one or a few tissue types. Some promoters are “inducible,” and achieve gene transcription under the influence of an inducer. Induction can occur, e.g., as the result of a physiologic response, a response to outside signals, or as the result of artificial manipulation. Some promoters respond to the presence of tetracycline; “rtTA” is a reverse tetracycline controlled transactivator. Such promoters are well known to those of skill in the art.

To bring a coding sequence under the control of a promoter, it is advantageous to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable promoters are generally obtained from viral genomes (e.g., polyoma, Simian Virus 40 (SV40), adenovirus, retroviruses, hepatitis-B virus, and cytomegalovirus) or heterologous mammalian genes (e.g. beta actin promoter). Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers also often contain response elements that mediate the regulation of transcription. Many enhancer sequences are now known from mammalian genes (globin, elastase, albumin, α-fetoprotein and insulin). However, an enhancer from a eukaryotic cell virus is preferably used for general expression. Suitable examples include the SV40 enhancer on the late side of the replication origin, the cytomegalovirus early promoter enhancer, the polyoma enhancer on the late side of the replication origin, and adenovirus enhancers.

In certain embodiments the promoter and/or enhancer region can act as a constitutive promoter and/or enhancer to maximize expression of the region of the transcription unit to be transcribed. In certain constructs the promoter and/or enhancer region is active in all eukaryotic cell types, even if it is only expressed in a particular type of cell at a particular time. A preferred promoter of this type is the CMV promoter. In other embodiments, the promoter and/or enhancer is tissue or cell specific.

In certain embodiments the promoter and/or enhancer region is inducible. Induction can occur, e.g., as the result of a physiologic response, a response to outside signals, or as the result of artificial manipulation. Such promoters are well known to those of skill in the art. For example, in some embodiments, the promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

### D. Host Cells

Host cells, and compositions and methods of making the disclosed proteins and polypeptides are provided.

### 1. In Vivo Methods

Host cells including the disclosed nucleic acid molecules are also provided. As discussed in more detail below, in some embodiments, particularly those where the polypeptides include a non-standard amino acid, can be carried out in vivo using a genomically recoded organism (GRO) or other host organism, nucleic acids encoding the orthogonal AARS and tRNA operably linked to one or more expression control sequences are introduced or integrated into cells or organisms. The heterologous mRNA encoding the polypeptide or protein of interest (e.g., the engineered fimbrial polypeptides) is introduced or integrated into host cells or organisms, and can also be linked to an expression control sequence.

a. Genomically Recoded Organisms

The host can be a genomically recoded organism (GRO). The GRO can be transformed or genetically engineered to express the orthogonal AARS-tRNA pair and the mRNA of interest. As discussed in more detail below, the AARS-tRNA pair and mRNA of interest transformed or transfected into the host expressed extrachromosomally, for example by plasmid(s) or another vector(s) or an episome, or can be integrated into the host's genome. The GRO host organism prior to transfection or integration of the AARS-tRNA pair can be referred to as a precursor or parental GRO.

Typically, the GRO is a cell or cells, preferably a bacterial strain, for example, an E. coli bacterial strain, wherein one or more codons has been replaced by a synonymous or even a non-synonymous codon. Because there are 64 possible 3-base codons, but only 20 canonical amino acids (plus stop codons), some amino acids are coded for by 2, 3, 4, or 6 different codons (referred to herein as “synonymous codons”). In a GRO, most or preferably all, of the instances of a particular codon are replaced with a synonymous (or non-synonymous) codon. Preferably, the GRO is recoded such that at least one codon is completely absent from the genome (also referred to as an eliminated codon). In some embodiments, two, three, four, five, six, seven, eight, nine, ten, or more codons are eliminated. Removal of a codon from the precursor GRO allows reintroduction of the deleted codon in a heterologous mRNA of interest. As discussed in more detail below, the reintroduced codon is typically dedicated to a non-standard amino acid, which in the presence of the appropriate orthogonal translation machinery, can be incorporated in the nascent peptide chain of during translation of the mRNA.

When a sense codon is eliminated, its elimination is preferably accompanied by mutation, or reduction or elimination of expression, of the cognate tRNA that decodes the codon during translation, reducing or eliminating the recognition of the codon by the tRNA. For example, the tRNA can be deleted from the organism, the tRNA can be mutated to recognized fewer or different codons (e.g., from recognizing AUA and AUC to just recognizing AUC), etc. In preferred embodiments, tRNAs that decode a particular codon(s) are deleted, as in some instances (due to Wobble effect), one tRNA decodes>1 codon (e.g., AGG, AGA).

When a nonsense codon is eliminated, its elimination is preferably accompanied by mutation, reduction, or deletion of the endogenous factor or factors. For example, release factor(s), associated with terminating translation at the nonsense codon (e.g., to reduce or eliminate expression of the release factor or change the recognition specificity of codons for the release factor).

In some embodiments, wherein the organism does not have or use certain codon(s), the unused (i.e., eliminated) codon may not be strictly considered sense or nonsense codons, but can nonetheless be utilized in the strategies discussed herein. For example, a host organism can be created by taking a codon an organism does not have or use, but can still be recognized (see. e.g., Krishnakumar, et al., Chembiochem., 14(15):1967-72 (2013). doi: 10.1002/cbic.201300444) and mutating its translation machinery, e.g., tRNA and/or factors such release factors, to have a greater specificity, thus creating an unassigned codon.

In some embodiments, a sense codon is reassigned as a nonsense codon. Typically a release factor that recognizes the reassigned nonsense codon is also expressed by such organisms.

Different organisms often show particular preferences for one of the several codons that encode the same amino acid, and some codons are considered rare or infrequent. Preferably, the replaced codon is one that is rare or infrequent in the genome. The replaced codon can be one that codes for an amino acid (i.e., a sense codon) or a translation termination codon (i.e., a stop codon). GRO that are suitable for use as host or parental strains for the disclosed systems and methods are known in the art, or can be constructed using known methods. See, for example, Isaacs, et al., Science, 333, 348-53 (2011), Lajoie, et al., Science 342, 357-60 (2013), Lajoie, et al., Science, 342, 361-363 (2013). Chin, et al., Nature, 569(7757):514-518 (2019). doi: 10.1038/s41586-019-1192-5, Ostrov, et al., Science, 353(6301):819-22 (2016). doi: 10.1126/science.aaf3639. See also the Sc2.0 project focused on synthesizing a new version of Saccharomyces cerevisiae refer to as Sc2.0.

In some embodiments, the eliminated codon is one that codes for a rare stop codon. In a particular embodiment, the GRO is one in which all instances of the UAG (TAG) codon have been removed and replaced by another stop codon (e.g., TAA, TGA), and preferably wherein release factor 1 (RF1; terminates translation at UAG and UAA) has also been deleted, eliminating translational termination at UAG codons (Lajoie, et al., Science 342, 357-60 (2013)). In a particular embodiment, the GRO is C321.Δ A [321 UAG→UAA conversions and deletion of prfA (encodes RF1)](genome sequence at GenBank accession CP006698), or a further modified strain thereof. In this GRO the UAG is eliminated. That is, UAG has been transformed from a nonsense codon (terminates translation). UAG is a preferred codon for elimination or recoding because it is the rarest codon in Escherichia coli MG1655 (321 known instances) and a rich collection of translation machinery capable of incorporating non-standard amino acids has been developed for UAG (Liu and Schultz, Annu. Rev. Biochem., 79:413-44 (2010), discussed in more detail below).

Stop codons include TAG (UAG), TAA (UAA), and TGA (UGA). Although recoding to UAG (TAG) is discussed in more detail above, it will be appreciated that either of the other stop codons (or any sense codon) can be elimination and optionally reintroduced using the same strategy. Accordingly, in some embodiments, a sense codon is eliminated, e.g., AGG or AGA to CGG, CGA, CGC, or CGG (arginine), e.g., as the principles can be extended to any set of synonymous or even non-synonymous codons, that are coding or non-coding. The foregoing is non-limiting example.

Similarly, the cognate translation machinery can be removed/mutated/deleted to remove natural codon function (e.g., nonsense codons UAG—RF1; UGA—RF2; tRNA corresponding to an eliminated sense codon, etc). The OTS system, particularly the antisense codon of the tRNA, can be designed to match a reintroduced codon, provided at least one codon remains eliminated. See also, Chin, et al., Nature, 569(7757):514-518 (2019). doi: 10.1038/s41586-019-1192-5, e.g., isoleucine, and Ostrov, et al., Science, 353(6301):819-822 (2016) DOI: 10.1126/science.aaf3639, which describes reducing the number of codons in E. coli from 64 to 57 by removing instances of the UAG stop codon and excising two arginine codons, two leucine codons, and two serine codons

Prokaryotes useful as GRO cells include, but are not limited to, gram negative or gram positive organisms such as E. coli or Bacilli, and although the most preferred host organism is a bacterial GRO, it will be appreciated the methods and compositions disclosed herein can be adapted for use on other host GRO organisms, including, but not limited to, eukaryotic cells, including e.g., yeast, fungi, insect, plant, animal, human, etc. cells, and, viruses.

GRO can have two, three, or more codons replaced with a synonymous codon. Such GRO allow for reintroduction of the two, three, or more deleted codons in a heterologous mRNA of interest, each dedicated to a different non-standard amino acid. Such GRO can be used in combination with the appropriate orthogonal translation machinery to produce polypeptides having two, three, or more different non-standard amino acids.

b. Other In Vivo Host Systems

Although a preferred host organism is a GRO, it will be appreciated that the methods and compositions disclosed herein can be adapted for use on other host organisms or in vitro. Other hosts and in vitro systems for translation are known in the art.

Suitable organisms include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems.

It will be understood by one of ordinary skill in the art that regardless of the system used (i.e. in vitro or in vivo), expression of genes encoding orthogonal AARS and tRNA will result in site specific incorporation of non-standard amino acids such as pAzF into the target polypeptides or proteins encoded by the specific heterologous mRNA transfected or integrated into the organism. Host cells are genetically engineered (e.g., transformed, transduced or transfected) with the vectors encoding orthogonal AARS, tRNA and heterologous mRNA which can be, for example, a cloning vector or an expression vector. The vector can be, for example, in the form of a plasmid, a bacterium, a virus, a naked polynucleotide, or a conjugated polynucleotide. The vectors are introduced into cells and/or microorganisms by standard methods including electroporation, infection by viral vectors, high velocity ballistic penetration by small particles with the nucleic acid either within the matrix of small beads or particles, or on the surface. Such vectors can optionally contain one or more promoter. A “promoter” as used herein is a DNA regulatory region capable of initiating transcription of a gene of interest.

Kits are commercially available for the purification of plasmids from bacteria, (see, e.g., GFX™ Micro Plasmid Prep Kit from GE Healthcare; STRATAPREP® Plasmid Miniprep Kit and STRATAPREP® EF Plasmid MIDIPREP Kit from Stratagene; GENELUTE™ HP Plasmid Midiprep and MAXIPREP Kits from Sigma-Aldrich, and, Qiagen plasmid prep kits and QIAfilter™ kits from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems.

Prokaryotes useful as host cells include, but are not limited to, gram negative or gram positive organisms such as E. coli or Bacilli. In a prokaryotic host cell, a polypeptide may include an N-terminal methionine residue to facilitate expression of the recombinant polypeptide in the prokaryotic host cell. The N-terminal Met may be cleaved from the expressed recombinant polypeptide. Promoter sequences commonly used for recombinant prokaryotic host cell expression vectors include lactamase and the lactose promoter system.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. To construct an expression vector using pBR322, an appropriate promoter and a DNA sequence are inserted into the pBR322 vector. Other commercially available vectors include, for example, T7 expression vectors from Invitrogen, pET vectors from Novagen and pALTER® vectors and PinPoint® vectors from Promega Corporation.

Yeasts useful as host cells include, but are not limited to, those from the genus Saccharomyces, Pichia, K. Actinomycetes and Kluyveromyces. Yeast vectors will often contain an origin of replication sequence, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Fleer et al., Gene, 107:285-195 (1991), in Li, et al., Lett Appl Microbiol. 40(5):347-52 (2005), Jansen, et al., Gene 344:43-51 (2005) and Daly and Hearn, J. Mol. Recognit. 18(2):119-38 (2005). Other suitable promoters and vectors for yeast and yeast transformation protocols are well known in the art.

Mammalian or insect host cell culture systems well known in the art can also be employed for producing proteins or polypeptides. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are well known in the art.

c. In Vitro Transcription/Translation

In some embodiments, the nucleic acids encoding AARS and tRNA are synthesized prior to translation of the target protein and are used to incorporate non-standard amino acids into a target protein in a cell-free (in vitro) protein synthesis system.

In vitro protein synthesis systems involve the use crude extracts containing all the macromolecular components (70S or 80S ribosomes, tRNAs, aminoacyl-tRNA synthetases, initiation, elongation and termination factors, etc.) required for translation of exogenous RNA. To ensure efficient translation, each extract must be supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors (Mg2+, K+, etc.).

In vitro protein synthesis does not depend on having a polyadenylated RNA, but if having a poly(A) tail is essential for some other purpose, a vector may be used that has a stretch of about 100 Å residues incorporated into the polylinker region. That way, the poly(A) tail is “built in” by the synthetic method. In addition, eukaryotic ribosomes read RNAs that have a 5′ methyl guanosine cap more efficiently. RNA caps can be incorporated by initiation of transcription using a capped base analogue, or adding a cap in a separate in vitro reaction post-transcriptionally.

Suitable in vitro transcription/translation systems include, but are not limited to, the rabbit reticulocyte system, the E. coli S-30 transcription-translation system, the wheat germ based translational system. Combined transcription/translation systems are available, in which both phage RNA polymerases (such as T7 or SP6) and eukaryotic ribosomes are present. One example of a kit is the TNT® system from Promega Corporation.

### 2. Orthogonal Translation System

a. Expression Strategies

Translation systems include most or all of the translation machinery of the host organism and additionally include a heterologous aminoacyl-tRNA synthetase (AARS)-rRNA pair (also referred to as an orthogonal translation system (OTS)) that can incorporate one or more non-standard amino acids into a growing peptide during translation of the heterologous mRNA. AARS are enzymes that catalyze the esterification of a specific cognate amino acid or its precursor to one or all of its compatible cognate tRNAs to form an aminoacyl-tRNA. An AARS can be specific for one non-standard amino acid, or can be polyspecific for two or more non-standard amino acids, canonical amino acids, or a combination thereof. The heterologous AARS used in the disclosed system typical can recognize, bind to, and transfer at least one non-standard amino acid to a cognate tRNA. Accordingly, the AARS can be selected by the practitioner based on the non-standard amino acid on interest. Some of the disclosed systems include two or more heterologous AARS.

tRNA is an adaptor molecule composed of RNA, typically about 76 to about 90 nucleotides in length that carries an amino acid to the protein synthetic machinery. Typically, each type of tRNA molecule can be attached to only one type of amino acid, so each organism has many types of tRNA (in fact, because the genetic code contains multiple codons that specify the same amino acid, there are many tRNA molecules bearing different anticodons which also carry the same amino acid). The heterologous tRNA used in the disclosed systems is one that can bind to the selected heterologous AARS and receive a non-standard amino acid to form an aminoacyl-tRNA. Because the transfer for the amino acid to the tRNA is dependent in-part on the binding of the tRNA to the AARS, these two components are typically selected by the practitioner based on their ability to interact with each other and participate in protein synthesis including the non-standard amino acid of choice in the host organism. Therefore, a selected heterologous AARS and tRNA are often referred to herein together as a heterologous AARS-tRNA pair, or an orthogonal translation system. Preferably, the heterologous AARS-tRNA pair does not cross-react with the existing host cell's pool of synthetases and tRNAs, or do so a low level (e.g., inefficiently), but is recognized by the host ribosome. Therefore, preferably the heterologous AARS cannot charge an endogenous tRNA with a non-standard amino acid (or does so a low frequency), and/or an endogenous AARS cannot charge the heterologous tRNA with a standard amino acid. Furthermore, preferably, the heterologous AARS cannot charge its paired heterologous tRNA with a standard amino acid (or does so at low frequency).

The heterologous tRNA also includes an anticodon that recognizes the codon of the codon in the heterologous mRNA that encodes the non-standard amino acid of choice. In the most preferred embodiment, the anticodon is one that hybridizes with a codon that is reduced or deleted in the host organism and reintroduced by the heterologous mRNA. For example, if the reduced or deleted codon is UAG (TAG), as in C321.Δ A, the heterologous tRNA anticodon is typically CUA.

In the disclosed expression systems can include at least one orthogonal pair is dedicated to incorporation of a non-standard amino acid into a polypeptide. However, the system may include two, three, or more orthogonal pairs, where one is dedicated to one non-standard amino acids, and one or more are dedicated to incorporation of one or more other non-standard amino acids.

The AARS-tRNA pair can be from an archaea, such as Methanococcus maripaludis, Methanocaldococcus jannaschii, Methanopyrus kandleri, Methanococcoides burtonii, Methanospirillum hungatei, Methanocorpusculum labreanum, Methanoregula boonei, Methanococcus aeolicus, Methanococcus vannieli, Methanosarcina mazei, Methanosarcina barkeri, Methanosarcina acetivorans, Methanosaeta thermophila, Methanoculleus marisnigri, Methanocaldococcus vulcanius, Methanocaldococcus fervens, or Methanosphaerula palustris, for can be variant evolved therefrom.

Suitable heterologous AARS-tRNA pairs for use in the disclosed systems and methods are known in the art. For example, Table 1 and the electronic supplementary information provided in Dumas, et al., Chem. Sci., 6:50-69 (2015), provide non-natural amino acids that have been genetically encoded into proteins, the reported mutations in the AARS that permit their binding to the non-natural amino acid, the corresponding tRNA, and a host organism in which the translation system is operational. See also Liu and Schultz, Annu. Rev. Biochem., 79:413-44 (2010) and Davis and Chin, Nat. Rev. Mol. Cell Biol., 13:168-82 (2012), which provide additional examples of AARS-tRNA pairs which can be used in the disclosed systems and methods. Preferred AARS with improved activity and specificity for the specific non-naturally occurring amino acids are disclosed and described in WO 2015/120287, which is specifically incorporated by reference herein in its entirety.

The AARS and tRNA can be provided separately, or together, for example, as part of a single construct. In a particular embodiment, the AARS-tRNA pair is evolved from a Methanocaldococcus jannaschii aminoacyl-tRNA synthetase(s) (AARS)/suppressor tRNA pairs and suitable for use in an E. coli host organism. See, for example, Young, J. Mol. Biol., 395(2):361-74 (2010), which describes an OTS including constitutive and inducible promoters driving the transcription of two copies of a M. jannaschii AARS gene in combination with a suppressor tRNA(CUA)(opt) in a single-vector construct.

During protein synthesis, tRNAs with attached amino acids are delivered to the ribosome by proteins called elongation factors (EF-Tu in bacteria, eEF-1 in eukaryotes), which aid in decoding the mRNA codon sequence. If the tRNA's anticodon matches the mRNA, another tRNA already bound to the ribosome transfers the growing polypeptide chain from its 3′ end to the amino acid attached to the 3′ end of the newly delivered tRNA, a reaction catalyzed by the ribosome. Accordingly, the heterologous AARS-tRNA pair should be one that can be processed by the host organism's elongation factor(s). Additional or alternatively, the system can include additional or alternative elongation factor variants or mutants that facilitate delivery of the heterologous aminoacyl-tRNA to the ribosome.

It will also be appreciated that methods of altering the anticodon of tRNA are known in the art. Any suitable tRNA selected for use in the disclosed systems and methods can be modified to hybridize to any desired codon. For example, although many of the heterologous tRNA disclosed here and elsewhere have a CUA anticodon, CUA can be substituted for another stop anticodon (e.g., UUA or UCA), or anticodon for any desired sense codon. The tRNA anticodon can be selected based on the GRO and the sequence of the heterologous mRNA as discussed in more detail above.

The OTS can also include mutated EF-Tu, in addition to AARS and tRNA, especially for bulky and/or highly charged NSAAs (e.g., phosphorylated amino acids) (Park, et al., Science, 333:1151-4 (2011)).

b. AARS

Methods of making AARS are provided in WO 2015/120287 (which is specifically incorporated by reference herein in its entirety), and variant AARS obtaining according to the methods, including, but not limited to those provided in WO 2015/120287 are provided and can be used in the disclosed methods. DNA sequence(s) can also be deduced from the amino acid sequence of the variant. Accordingly, nucleic acid sequences encoding variant AARS are also provided.

The precise percentage of similarity between sequences that is useful in establishing sequence identity varies with the nucleic acid and protein at issue, but as little as 25% sequence similarity is routinely used to establish sequence identity. Higher levels of sequence similarity, e.g., at least 30%, 40%, 50%, 60%, 70%, 80%, 90%, 95% or 99% or more can also be used to establish sequence identity. Therefore, in some embodiments, the variant includes at least 30%, 40%, 50%, 60%, 70%, 80%, 90%, 95% or 99% or more sequence identity with the parent AARS.

Variant AARS of a parent M. jannaschii AARS referred to pAcF AARS (pAcFRS) (Young, et al., J Mol Biol, 395:361-74 (2010)) are provided. The amino acid sequence for pAcFRS is

(SEQ ID NO: 24)
MDEFEMIKRNTSEIISEEELREVLKKDEKSALIGFEPSGKIHLGHYLQIKK
MIDLQNAGFDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVAVGGMEQRKIHMLARELLPKKVVCIHNPVLTGLDGEGK
MSSSKGNFIAVDDSPEEIRAKIKKAYCPAGVVEGNPIMEIAKYFLEYPLTI
KRPEKFGGDLTVNSYEELESLFKNKELHPMRLKNAVAEELIKILEPIRKR
L.

The nucleic acid sequence for a cognate tRNA of SEQ ID NO:24 is CCGGCGGTAGTTCAGCAGGGCAGAACGGCGGACTCTAAATCCGCATGGCAG GGGTTCAAATCCCCTCCGCCGGACCA (SEQ ID NO:25). This tRNA can also be a cognate tRNA for the variant AARS described in more detail below.

Variants of pAcFRS have one or more mutations relative to SEQ ID NO:1, and typically have altered specificity and/or activity toward one or more non-standard amino acids and/or altered specificity and/or activity toward a paired tRNA relative to the protein of SEQ ID NO:24. In some embodiments, the variant includes at least 30%, 40%, 50%, 60%, 70%, 80%, 90%, 95% or 99% or more sequence identity with the parent AARS, or a functional fragment thereof.

The variants typically have one or more substitution mutations in the non-standard amino acid (amino acid ligand) binding pocket of SEQ ID NO:24, the tRNA anticodon recognition interface of SEQ ID NO:24, or a combination thereof. For example, the variants can have a substitution mutation at one or more of amino acid positions 65, 107, 108, 109, 158, 159, 162, 167, 257, and 261 of SEQ ID NO:24 relative to the N-terminal methionine of SEQ ID NO:24.

Exemplary variants are provided below and have nsAA specificities at least as provided.

pAcFRS.1
(polyspecificity for at least pAcF, pAzF, StyA,
4IF, 4BrF, 4CIF, 4MeF, 4Cf3F, MeY, 4NO2F, 4BuF,
BuY, 2NaA, PheF):
(SEQ ID NO: 26)
MDEFEMIKRNTSEIISEEELREVLKKDEKSALIGFEPSGKIHLGHY
LQIKKMIDLQNAGEDIIILLADLHAYLNQKGELDEIRKIGDYNKKVFEAMG
LKAKYVYGSEFOLDKDYTLNVYRLALKTTLKRARRSMELIAREDENPKVAE
VIYPIMQVNGCHYRGVDVDVGGMEQRKIHMLARELLPKKVVCIHNPVLTGL
DGEGKMSSSKGNFIAVDDSPEEIRAKIKKAYCPAGVVEGNPIMEIAKYFLE
YPLTIKRPEKFGGDLTVNSYEELESLFKNKELHPMRLKNAVAEELIKILEP
IRKRL;
pAcFRS.t1
(polyspecificity for at least pAcF, pAzF, StyA):
(SEQ ID NO: 27)
MDEFEMIKRNTSEIISEEELREVLKKDEKSALIGFEPSGKIHLGHYLQIKK
MIDLQNAGEDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVAVGGMEQRKIHMLARELLPKKVVCIHNPVLTGLDGEGK
MSSSKGNFIAVDDSPEEIRAKIKKAYCPAGVVEGNPIMEIAKYFLEYPLTI
KGPEKFGGDLTVNSYEELESLFKNKELHPMRLKNAVAEELIKILEPIRKR
L;
pAcFRS.t2
(polyspecificity for at least pAcF, pAzF, StyA):
(SEQ ID NO: 28)
MDEFEMIKRNTSEIISEEELREVLKKDEKSALIGFEPSGKIHLGHYLQIKK
MIDLQNAGFDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVAVGGMEQRKIHMLARELLPKKVVCIHNPVLTGLDGEGK
MSSSKGNFIAVDDSPEEIRAKIKKAYCPAGVVEGNPIMEIAKYFLEYPLTI
KCPEKEGGDLTVNSYEELESLFKNKELHPMRLKNAVAEELIKILEPIRKR
L;
pAcFRS.1.t1
(polyspecificity for at least pAcF, pAzF, StyA,
4IF, 4BrF, 4CIF, 4MeF, 4Cf3F, MeY, 4NO2F, 4BuF,
BuY, 2NaA, PheF):
(SEQ ID NO: 29)
MDEFEMIKRNTSEIISEEELREVLKKDEKSALIGFEPSGKIHLGHYLQIKK
MIDLQNAGFDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVDVGGMEQRKIHMLARELLPKKVVCIHNPVLTGLDGEGK
MSSSKGNFIAVDDSPEEIRAKIKKAYCPAGVVEGNPIMEIAKYFLEYPLTI
KGPEKFGGDLTVNSYEELESLFKNKELHPMRLKNAVAEELIKILEPIRKR
L;
pAcFRS.1.t2
(polyspecificity for at least pAcF, pAzF, StyA,
4IF, 4BrF, 4CIF, 4MeF, 4Cf3F, MeY, 4NO2F, 4BuF,
BuY, 2NaA, PheF):
(SEQ ID NO: 30)
MDEFEMIKRNTSEIISEEELREVLKKDEKSALIGFEPSGKIHLGHYLQIKK
MIDLQNAGFDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVDVGGMEQRKIHMLARELLPKKVVCIHNPVLTGLDGEGK
MSSSKGNFIAVDDSPEEIRAKIKKAYCPAGVVEGNPIMEIAKYFLEYPLTI
KCPEKEGGDLTVNSYEELESLFKNKELHPMRLKNAVAEELIKILEPIRKR
L;
pAcFRS.2
(polyspecificity for at least pAcF, pAzF, StyA,
4IF, 4BrF, 4CIF, 4MeF, 4Cf3F, MeY, 4NO2F,
4BuF, BuY, 2NaA, PheF).
(SEQ ID NO: 31)
MDEFEMIKRNTSEIISEEELREVLKKDEKSALIGFEPSGKIHLGHYLQIKK
MIDLONAGFDIIIVLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVDVGGMEQRKIHMLARELLPKKVVCIHNPVLTGLDGEGK
MSSSKGNFIAVDDSPEEIRAKIKKAYCPAGVVEGNPIMEIAKYFLEYPLTI
KRPEKFGGDLTVNSYEELESLFKNKELHPMRLKNAVAEELIKILEPIRKR
L;
pAcFRS.2.t1
(polyspecificity for at least pAcF, pAzF, StyA,
4IF, 4BrF, 4CIF, 4MeF, 4Cf3F, MeY, 4NO2F, 4BuF,
BuY, 2NaA, PheF)
(SEQ ID NO: 32)
MDEFEMIKRNTSEIISEEELREVLKKDEKSALIGFEPSGKIHLGHYLQIKK
MIDLQNAGFDIIIVLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVDVGGMEQRKIHMLARELLPKKVVCIHNPVLTGLDGEGK
MSSSKGNFIAVDDSPEEIRAKIKKAYCPAGVVEGNPIMEIAKYFLEYPLTI
KGPEKFGGDLTVNSYEELESLFKNKELHPMRLKNAVAEELIKILEPIRKR
L;
pAcFRS.2.t2
(polyspecificity for at least pAcF, pAzF, StyA,
4IF, 4BrF, 4CIF, 4MeF, 4Cf3F, MeY, 4NO2F, 4BuF,
BuY, 2NaA, PheF):
(SEQ ID NO: 33)
MDEFEMIKRNTSEIISEEELREVLKKDEKSALIGFEPSGKIHLGHYLQIKK
MIDLONAGFDIIIVLADLHAYLNQKGELDEIRKIGDYNKKVFEAMGLKAKY
VYGSEFQLDKDYTLNVYRLALKTTLKRARRSMELIAREDENPKVAEVIYPI
MQVNGCHYRGVDVDVGGMEQRKIHMLARELLPKKVVCIHNPVLTGLDGEGK
MSSSKGNFIAVDDSPEEIRAKIKKAYCPAGVVEGNPIMEIAKYFLEYPLTI
KCPEKEGGDLTVNSYEELESLFKNKELHPMRLKNAVAEELIKILEPIRKR
L;
pAzFRS.1 (specific for pAzF):
(SEQ ID NO: 34)
MDEFEMIKRNTSEIISEEELREVLKKDEKSALIGFEPSGKIHLGHYLQIKK
MIDLQNAGFDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNVMHYDGVDVYVGGMEQRKIHMLARELLPKKVVCIHNPVLTGLDGEGK
MSSSKGNFIAVDDSPEEIRAKIKKAYCPAGVVEGNPIMEIAKYFLEYPLTI
KRPEKFGGDLTVNSYEELESLFKNKELHPMRLKNAVAEELIKILEPIRKR
L;
pAzFRS.1.t1
(specific for pAzF):
(SEQ ID NO: 35)
MDEFEMIKRNTSEIISEEELREVLKKDEKSALIGFEPSGKIHLGHYLQIKK
MIDLONAGEDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNVMHYDGVDVYVGGMEQRKIHMLARELLPKKVVCIHNPVLTGLDGEGK
MSSSKGNFIAVDDSPEEIRAKIKKAYCPAGVVEGNPIMMEIAKYFLEYPLT
IKGPEKFGGDLTVNSYEELESLFKNKELHPMRLKNAVAEELIKILEPIRKR
L;
pAzFRS.1.t2 (specific for pAzF):
(SEQ ID NO: 36)
MDEFEMIKRNTSEIISEEELREVLKKDEKSALIGFEPSGKIHLGHYLQIKK
MIDLQNAGEDIIILLADLHAYLNQKGELDEIRKIGDYNKKVFEAMGLKAKY
VYGSEFQLDKDYTLNVYRLALKTTLKRARRSMELIAREDENPKVAEVIYPI
MQVNVMHYDGVDVYVGGMEQRKIHMLARELLPKKVVCIHNPVLTGLDGEGK
MSSSKGNFIAVDDSPEEIRAKIKKAYCPAGVVEGNPIMMEIAKYFLEYPLT
IKCPEKEGGDLTVNSYEELESLFKNKELHPMRLKNAVAEELIKILEPIRKR
L;
pAzRS.2
(polyspecific for at least pAcF, pAzF, StyA,
4IF, 4BrF, 4CIF, 4MeF, 4Cf3F, MeY, 4NO2F,
4BuF, BuY, 2NaA, PheF):
(SEQ ID NO: 37)
MDEFEMIKRNTSEIISEEELREVLKKDEKSALIGFEPSGKIHLGHYLQIKK
MIDLQNAGEDIIILLADLHAYLNOKGELDEIRKIGDYNKKVFEAMGLKAKY
VYGSTYMLDKDYTLNVYRLALKTTLKRARRSMELIAREDENPKVAEVIYPI
MQVNGCHYRGVDVAVGGMEQRKIHMLARELLPKKVVCIHNPVLTGLDGEGK
MSSSKGNFIAVDDSPEEIRAKIKKAYCPAGVVEGNPIMEIAKYFLEYPLTI
KRPEKFGGDLTVNSYEELESLFKNKELHPMRLKNAVAEELIKILEPIRKR
L;
pAzRS.2.t1
(polyspecific for at least pAcF, pAzF, StyA,
4IF, 4BrF, 4CIF, 4MeF, 4Cf3F, MeY, 4NO2F, 4BuF,
BuY, 2NaA, PheF):
(SEQ ID NO: 38)
MDEFEMIKRNTSEIISEEELREVLKKDEKSALIGFEPSGKIHLGHYLQIKK
MIDLQNAGFDIIILLADLHAYLNOKGELDEIRKIGDYNKKVFEAMGLKAKY
VYGSTYMLDKDYTLNVYRLALKTTLKRARRSMELIAREDENPKVAEVIYPI
MQVNGCHYRGVDVAVGGMEQRKIHMLARELLPKKVVCIHNPVLTGLDGEGK
MSSSKGNFIAVDDSPEEIRAKIKKAYCPAGVVEGNPIMEIAKYFLEYPLTI
KGPEKFGGDLTVNSYEELESLFKNKELHPMRLKNAVAEELIKILEPIRKR
L;
and
pAzRS.2.t2
(polyspecific for at least pAcF, pAzF, StyA, 4IF,
4BrF, 4CIF, 4MeF, 4Cf3F, MeY, 4NO2F, 4BuF, BuY,
2NaA, PheF):
(SEQ ID NO: 39)
MDEFEMIKRNTSEIISEEELREVLKKDEKSALIGFEPSGKIHLGHYLQIKK
MIDLQNAGFDIIILLADLHAYLNOKGELDEIRKIGDYNKKVFEAMGLKAKY
VYGSTYMLDKDYTLNVYRLALKTTLKRARRSMELIAREDENPKVAEVIYPI
MQVNGCHYRGVDVAVGGMEQRKIHMLARELLPKKVVCIHNPVLTGLDGEGK
MSSSKGNFIAVDDSPEEIRAKIKKAYCPAGVVEGNPIMEIAKYFLEYPLTI
KCPEKEGGDLTVNSYEELESLFKNKELHPMRLKNAVAEELIKILEPIRKR
L.

The position and domain of the mutation in each of SEQ ID NO:26-39 relative to SEQ ID NO:24 is provided in Table 1 below. Variants having any combination of the mutations disclosed in Table 1 are also specifically provided.

TABLE 1
Annotations of specific mutations in AARS variants
(mutations in evolved synthetases are annotated
with respect to the progenitor pAcFRS variant)

| | | Evolved for |
|---|---|---|
| Annotation | Mutant | nsAA/tRNA |
| pAcFRS | pAcFRS (Young, et al., J Mol | — |
| | Biol, 395: 361-74 (2010)) | |
| pAzFRS | pAzFRS (Schultz, et al., J Am | — |
| | Chem Soc, 128: 13984-5 (2006)) | |
| pAcFRS.1 | A167D | pAcF |
| pAcFRS.t1 | R257G | tRNA |
| pAcFRS.t2 | R257C, F261E | tRNA |
| pAcFRS.1.t1 | A167D, R257G | pAcF + tRNA |
| pAcFRS.1.t2 | A167D, R257C, F261E | pAcF + tRNA |
| pAzFRS.1 | D158V, I159M, L162D, A167Y | pAzF |
| pAzFRS.1.t1 | D158V, I159M, L162D, A167Y, | pAzF + tRNA |
| | R257G | |
| pAzFRS.1.t2 | D158V, I159M, L162D, A167Y, | pAzF + tRNA |
| | R257C, F261E | |
| pAzFRS.2 | E107T, F108Y, Q109M | pAzF |
| pAzFRS.2.t1 | E107T, F108Y, Q109M, R257G | pAzF + tRNA |
| pAzFRS.2.t2 | E107T, F108Y, Q109M, R257C, | pAzF + tRNA |
| | F261E | |
| pAcFRS.2 | L65V, A167D | pAcF |
| pAcFRS.2.t1 | L65V, A167D, R257G | pAcF + tRNA |
| pAcFRS.2.t2 | L65V, A167D, R257C, F261E | pAcF + tRNA |

### E. Methods Making Polypeptides

The methods making polypeptides such as the disclosed engineered fimbrial polypeptides are provided. Such methods can include chemical synthesis or by recombinant production in a host cell. To recombinantly produce the polypeptides, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the polypeptide. Useful prokaryotic and eukaryotic systems for expressing and producing polypeptides are well known in the art include, for example, Escherichia coli strains such as BL-21, and cultured mammalian cells such as CHO cells.

Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin or by metabolic selection using the Glutamine Synthetase-NSO system). The transfected cells can be cultured such that the polypeptide of interest is expressed, and the polypeptide can be recovered from, for example, the cell culture supernatant or from lysed cells. Alternatively, polypeptides can be made using in vitro transcription.

Polypeptides can be isolated using, for example, chromatographic methods such as DEAE ion exchange, gel filtration, hydroxylapatite chromatography, affinity chromatography, ion exchange chromatography, and hydrophobic interaction chromatography. In some embodiments, polypeptides can be engineered to contain an additional domain containing amino acid sequence that allows the polypeptides to be captured onto an affinity matrix.

As introduced above, the method can involve using an orthogonal AARS-tRNA pair in the translation process for introduction of non-standard amino acids. As discussed above, the AARS preferentially aminoacylates its cognate tRNA with a non-naturally occurring amino acid. The resulting aminoacyl-tRNA recognizes at least one codon in the mRNA for the target protein, such as a stop codon. An elongation factor (such as EF-Tu in bacteria) mediates the entry of the aminoacyl-tRNA into a free site of the ribosome. If the codon-anticodon pairing is correct, the elongation factor hydrolyzes guanosine triphosphate (GTP) into guanosine diphosphate (GDP) and inorganic phosphate, and changes in conformation to dissociate from the tRNA molecule. The aminoacyl-tRNA then fully enters the A site, where its non-standard amino acid is brought near the P site's polypeptide and the ribosome catalyzes the covalent transfer of the non-standard amino acid onto the polypeptide.

In some embodiments, as discussed above, the resulting polypeptides can be modified to include a further functional moiety or moieties using e.g., copper(J)-catalyzed azide-alkyne Huisgen cycloaddition (click-chemistry).

The resulting polypeptides can be isolated, purified, or otherwise enriched using methods known in the art, and discussed in more detail below.

In some embodiments, the heterologous AARS, its cognate tRNA, or more preferably both, are integrated into the host genome. Although suitable AARS are known in the art, in the most preferred embodiments, the AARS is a variant AARS that has improved binding to its cognate tRNA, its non-standard amino acid(s), or both compared to a known AARS. Exemplary variant AARS are discussed in more detail below.

The methods of making polypeptide are typically capable of producing polypeptides having a greater number of instances of non-standard amino acids and/or a greater yield of the desired polypeptide than the same or similar polypeptide made using conventional compositions, systems, and methods.

### F. Methods of Making Pili

Methods of making and isolating pili are known in the art (Korhonen et al., Infection and Immunity 27, 569-575 (1980)) and exemplified below, and can be adapted for use with the disclosed compositions and methods. For example, cells can be grown in pili producing conditions, such as in stagnant media without shaking for pili with standard amino acid substitutions made in the genome or in media with shaking supplemented with non-standard amino acid for pili with amino acid substitutions incorporated on a plasmid, and spun down and resuspended in ethanolamine (ETA), e.g., pH 10.5. Cells can be vortexed to shear pili from the cells, after which the cells can be centrifuged to collect the cells, keeping the supernatant and discarding the pellet. The supernatant can then be centrifuged to remove remaining contaminants, keeping the supernatant and discarding the debris pellet. Saturated ammonium sulfate (SAS) can be added to the supernatant to precipitate out pili proteins. The solution can then by centrifuged to collect the precipitated pili. The pili pellet can be resuspended, e.g., ETA, e.g., pH 10.5. To this sample, MgCl₂ can be added to again precipitate out pili. The solution can be centrifuged to pellet the pili. The supernatant can be discarded, and the pili pellet resuspended and solubilized ETA e.g., pH 10.5.

### G. Methods of Making Pili Networks

Methods of making pili networks are also provided. The networks can be 1D, 2D, or 3D ordered or unordered structures.

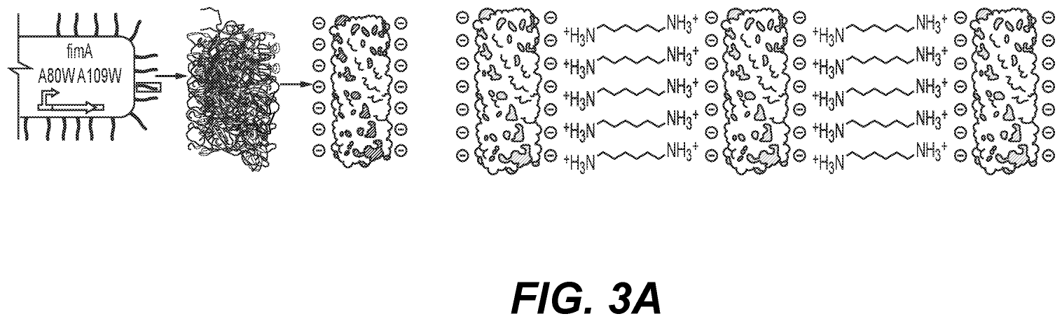

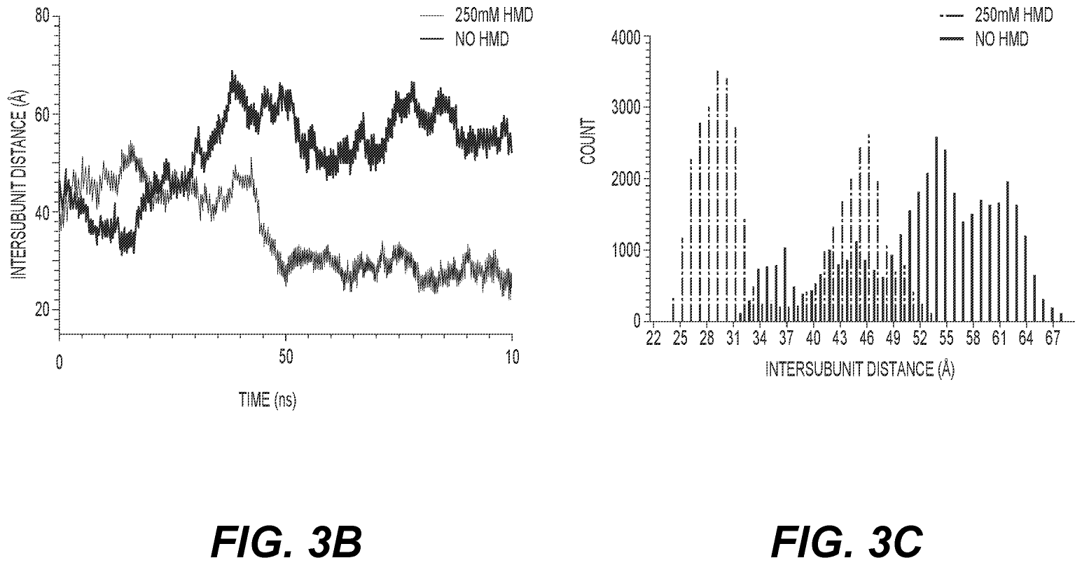

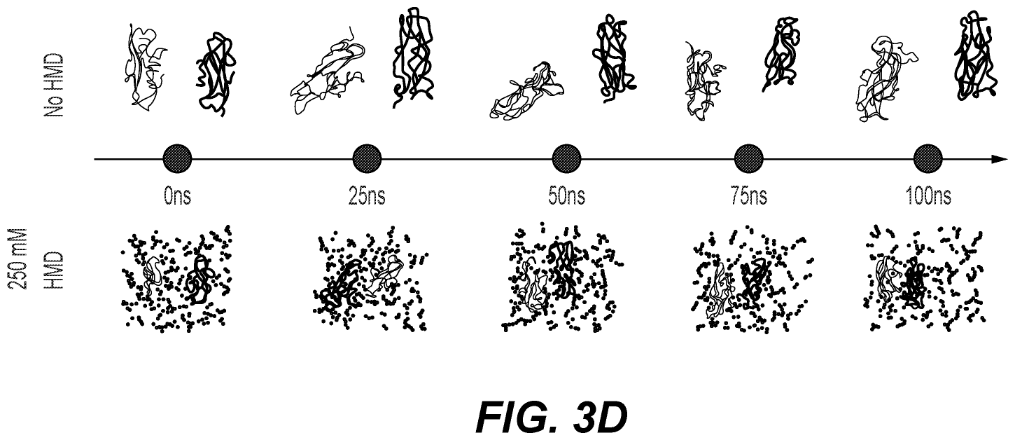

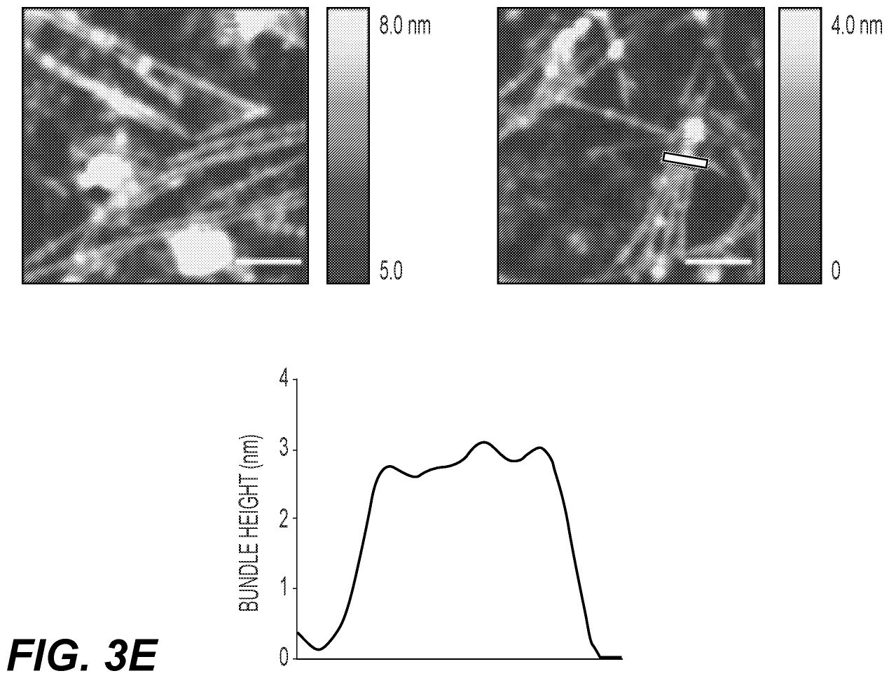

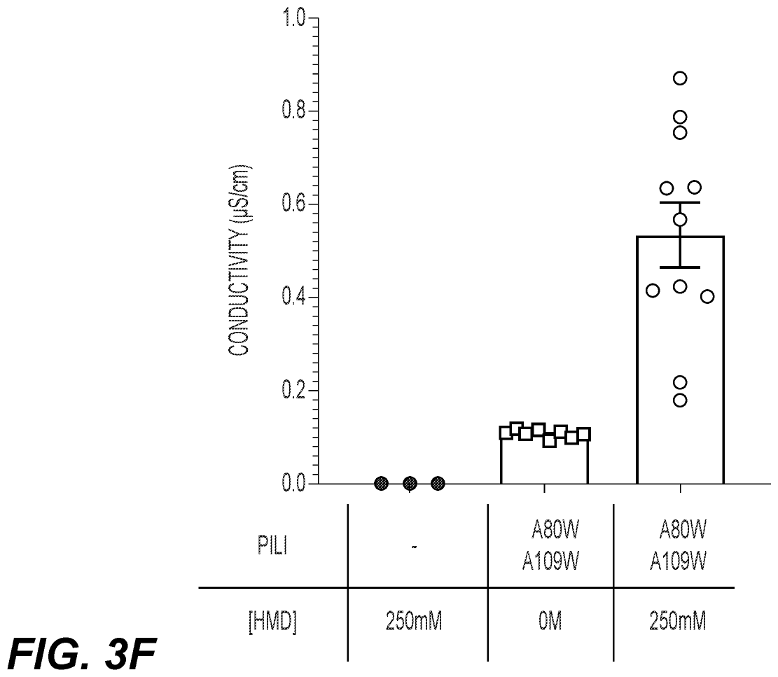

For example, the disclosed networks can be structured filament nanostructure formed by utilizing an inducer to align conductive pili into 2D or 3D bundled lattices through molecular recognition-based self-assembly (FIG. 3A). For example, a study demonstrated the ability to create different nanostructures by modulating the identity and concentration of inducer (Cao, et al., Angewandte Chemie (International ed. in English) 50, 6264 (2011), which is specifically incorporated by reference herein in its entirety), and can be utilized to prepare pili networks formed of the engineered fimbrial polypeptides disclosed herein. Suitable inducers include, but are not limited to, hexamethylenediamine (HMD), pimelic acid, and 1,3-propanedisulfonic acid. Inducers typically have either positively or negatively charged distal ends connected by a long central carbon chain.

Previous work shows that concentrations of the inducers in pili solutions can control whether the self-assembled nanostructures are 1D bundles, 2D double-layer lattices, or 3D multilayer lattices. High concentrations of positively charged inducers (e.g., hexamethylenediamine) can induce the self-assembly of pili into bundles through electrostatic interactions (e.g., within 1 h). Double-layer pili lattices can be formed in a lower concentration hexamethylenediamine solution after a longer incubation time (e.g., three days). In this case, only a proper concentration of hexamethylenediamine could induce the formation of double-layer pili lattices. If the concentration was too high, only bundles were produced; if the concentration was too low, no assemblies were observed. 3D multilayer pili lattices were not observed to be induced by hexamethylenediamine solution. They were formed in the presence of lower concentration (e.g., 80 mM) of pimelic acid (seven days) or 1,3-propanedisulfonic acid (20 days), both of which have negative charges (COOH or SO3H) at two distal ends. These inducer molecules could not electrostatically attract pili like hexamethylenediamine. Since they could not interact with pili directly, the nucleation and growth of pili lattices were slower. Compared to 1,3-propanedisulfonic acid, pimelic acid has a better precipitating efficiency, so the growth rate of pili in its solution (seven days) was faster than in 1,3-propanedisulfonic acid solution (20 days).

In the experiments below, HMD (250 mM) was used to create 2D bundled lattices, to investigate the effect of self-assembly on pili material conductivity. HMD is positively charged at both ends and thus is able to promote alignment of negatively-charged pili (FIG. 3A) into larger structures.

In other embodiments, magnetic beads can be attached to the pili, and magnetism can be used to align the molecules. See, e.g., Cao, et al., Angew Chem Int Ed Engl., 52(45):11750-4 (2013) doi: 10.1002/anie.201303854, which is specifically incorporated by reference herein in its entirety.

The disclosed invention can be further understood by the following numbered paragraphs:
1. A fimbrial polypeptide including one or more mutations with an aromatic amino acid relative to the corresponding wildtype fimbrial protein.
2. The fimbrial polypeptide of paragraph 1, wherein the substitution(s) include one or more additions and/or substitutions.
3. The fimbrial polypeptide of paragraphs 1 or 2, wherein aromatic amino acid is selected from phenylalanine, tyrosine, histidine, and tryptophan.
4. The fimbrial polypeptide of any one of paragraphs 1-3, wherein the aromatic amino acid is a non-standard amino acid.
5. The fimbrial polypeptide of any one of paragraphs 1-4, wherein the non-standard amino acid is a substrate for a click chemistry reaction.
6. The fimbrial polypeptide of paragraph 5, wherein the click chemistry reaction includes copper(I)-catalyzed azide-alkyne cycloaddition (CuAAC), strain-promoted azide-alkyne cycloaddition (SPAAC), strain-promoted alkyne-nitrone cycloaddition (SPANC); or strained alkene reactions optionally selected from alkene-azide [3+2]cycloaddition, the alkene-tetrazine inverse-demand Diels-Alder cycloaddition, and the alkene-tetrazole photoclick reaction
7. The fimbrial polypeptide of any one of paragraphs 4-6, wherein the non-standard amino acid(s) is propargyloxy-phenylalanine (PrOF), p-azido-1-phenylalanine (pAzF), 3-(2-Naphthyl)-L-alanine (2NaA), 4-Chloro-phenylalanine, 4-bromo-phenylalanine, para-acetyl-phenylalanine, para-amino-phenylalanine, 4-Iodo-phenylalanine, phenyl-L-phenylalanine, O-2-azidoethyl-tyrosine, para-azidomethyl-phenylalanine, 4-propargyloxy-1-phenylalanine (pPR), pAcF, StyA, 4IF, 4BrF, 4ClF, 4MeF, 4Cf3F, MeY, 4NO2F, 4BuF, BuY, and/or PheF.
8. The fimbrial polypeptide of any one of paragraphs 1-7, wherein the aromatic amino acid(s) includes a metal particle, optionally a nanoparticle, or a heme group conjugated thereto.
9. The fimbrial polypeptide of paragraph 8, wherein the metal is gold.
10. The fimbrial polypeptide of paragraph 9, wherein the aromatic amino acid is PrOF.
11. The fimbrial polypeptide of any one of paragraphs 1-9 including two or more mutations in close enough proximity to each other to facilitate efficient electron transfer, optionally<15 Å apart, in an assembled pilus formed thereof.
12. The fimbrial polypeptide of any one of paragraphs 1-11, wherein the wildtype protein is a FimA, optionally E. coli FimA, optionally wherein the E. coli FimA includes the amino acid sequence of SEQ ID NO:1.
13. The fimbrial polypeptide of any one of paragraphs 1-12, including at least 70% sequence identity to the wildtype protein.
14. The fimbrial polypeptide of paragraphs 12 or 13 including mutations at one or more of A80, H82, and A109 relative to the wildtype protein.
15. The fimbrial polypeptide of paragraph 14 including phenylalanine, tyrosine, histidine, and/or tryptophan substitutions at one or more of A80, H82, and A109 relative to the wildtype protein, optionally, wherein the mutation(s) include or consist of A80F, A109F, A80Y, A80 W, A109 W, A109Y, A80F A109F (double mutant), A80Y A109Y (double mutant), or A80 W A109 W (double mutant).
16. The fimbrial polypeptide of paragraph 14, including propargyloxy-phenylalanine (PrOF), p-azido-1-phenylalanine (pAzF), 3-(2-Naphthyl)-L-alanine (2NaA), 4-Chloro-phenylalanine, 4-bromo-phenylalanine, para-acetyl-phenylalanine, para-amino-phenylalanine, 4-Iodo-phenylalanine, phenyl-L-phenylalanine, O-2-azidoethyl-tyrosine, para-azidomethyl-phenylalanine, 4-propargyloxy-1-phenylalanine (pPR), pAcF, StyA, 4IF, 4BrF, 4ClF, 4MeF, 4Cf3F, MeY, 4NO2F, 4BuF, BuY, and/or PheF substitutions at one or more of A80, H82, and A109 relative to the wildtype protein, optionally, wherein the mutation(s) include or consist of PrOF substitution at A109, or pAzF or 2NaA pAzF or 2NaA substitution at A80;
optionally wherein the aromatic amino acids include a gold nanoparticle conjugate thereto.
17. A pilus including a plurality of the fimbrial polypeptide of any one of paragraphs 1-16.
18. A bundle of pili including a plurality of the pilus of paragraph 17.
19. The bundle of pili of paragraph 18, wherein the bundle is a 1D, 2D, or 3D bundle.
20. The bundle of pili of paragraphs 18 or 19, wherein the bundle is ordered.
21. The bundle of pili of any one of paragraphs 18-20 including a lattice structure.
22. The bundle of pili of any one of paragraphs 18-21 assembled with an inducer.
23. The bundle of pili of paragraph 20, wherein the inducer is hexamethylenediamine (HMD), pimelic acid, and 1,3-propanedisulfonic acid.
24. The fimbrial polypeptide, pilus, or bundle of pili of any one of paragraphs 1-23, wherein the fimbrial polypeptide, pilus, or bundle of pili is electrically conductive.
25. The fimbrial polypeptide, pilus, or bundle of pili of paragraph 24, wherein the fimbrial polypeptide, pilus, or bundle of pili is more conductive than the corresponding wildtype fimbrial polypeptide, pilus, or bundle of pili.
26. An electrical circuit including the fimbrial polypeptide, pilus, or bundle of pili of any one of paragraphs 1-25.
27. A device including fimbrial polypeptide, pilus, bundle of pili, or electrical circuit of any one of paragraphs 1-26.
28. The device of paragraph 27, wherein the device is a sensor, transistor, capacitor, electronic prosthetics, implantable electrode, flexible electronic, energy storage, soft robotics, computing, or information storage.
29. A system including the device of paragraph 28.
30. The electrical circuit, device, or system of any one of paragraphs 26-29, wherein the fimbrial polypeptide, pilus, or bundle of pili serves as the conductive element of the circuit, device, or system.
31. A method of making a fimbrial polypeptide including one or more iterations of an aromatic non-standard amino acid including expressing a messenger RNA (mRNA) encoding the fimbrial polypeptide in a system including:
an orthogonal translation system (OTS) including a nucleic acid sequence encoding an aminoacyl tRNA synthetase (AARS) and its cognate tRNA operably linked to expression control sequences and transformed, transfected, or integrated into a genomically recoded organism (GRO) with at least one codon reduced or absent from its genome, and
a plurality of the non-standard amino acid,
wherein the mRNA includes a nucleic acid sequence including at least one iteration of the codon deleted from the GRO,
wherein the AARS can charge the tRNA with non-standard amino acid,
and wherein the tRNA includes and anticodon that can bind to the codon reduced or absent from the GRO.
32. A method of making pili including making the fimbrial polypeptide according to the method of paragraph 31 in a prokaryotic host, optionally wherein the prokaryotic host is E. coli, and isolating pili formed by the host.
33. A method of forming an ordered bundle of pili including isolating pili made according to the method of paragraph 32, and contacting the pili with an inducer, optionally wherein the inducer is HMD.
34. A method of using the pili of paragraph 32 or the bundle of pili of paragraph 33 conduct electricity including connecting to the pili or bundle of pili to two electrode an applying electricity to one of the electrodes.

### EXAMPLES

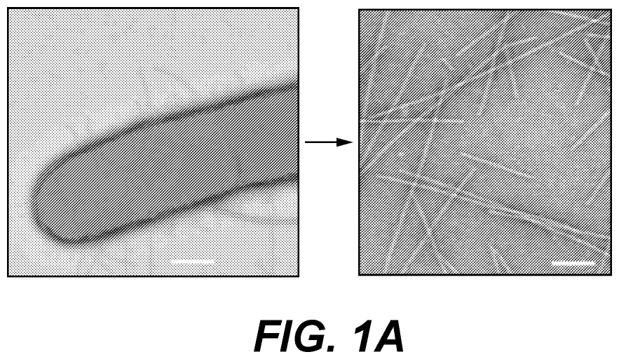

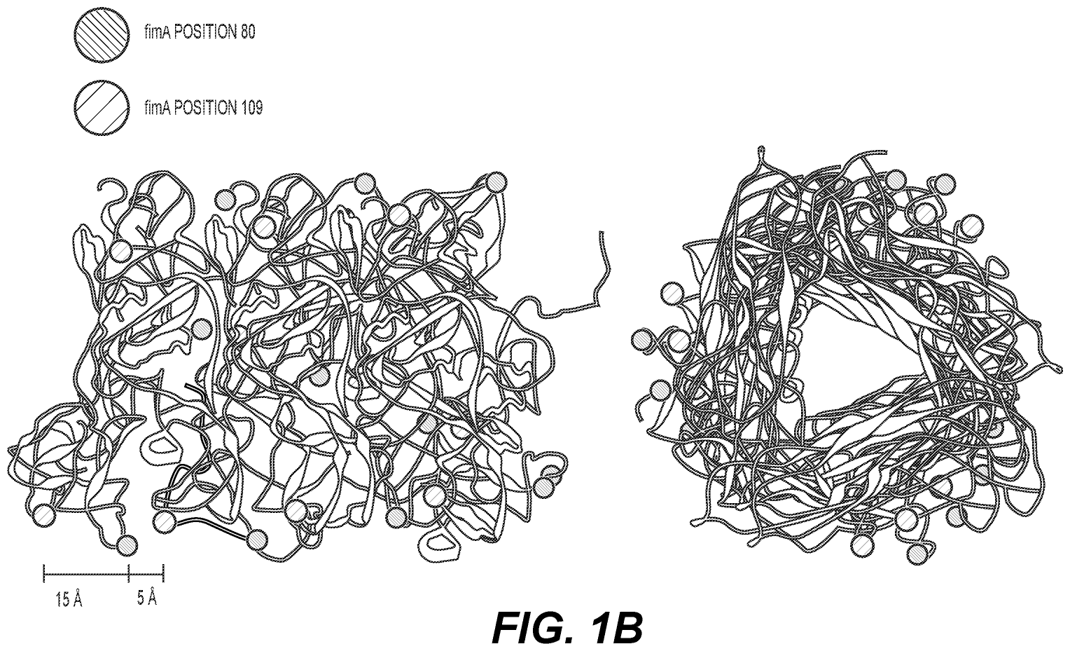

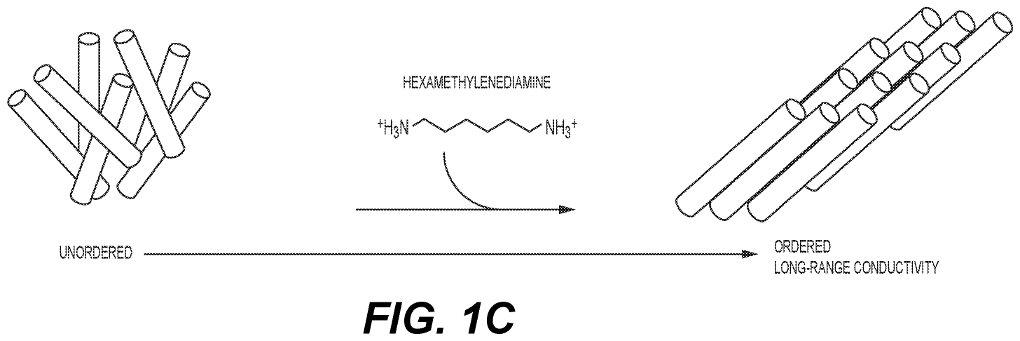

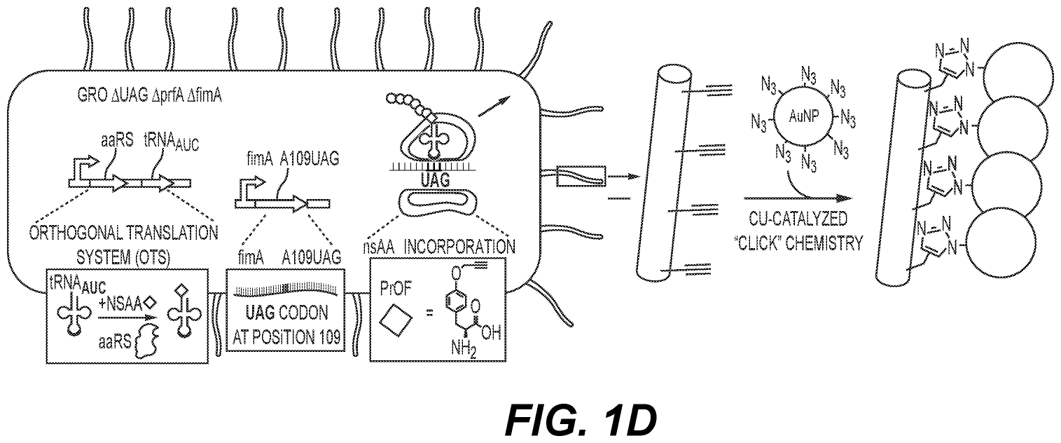

The experiments provided below address challenges of using pili in biomaterials by pursuing three strategies that demonstrate large-scale production of conductive pili proteins with tunable electronic properties. First, aromatic amino acid mutations were strategically encoded into E. coli type 1 pili (FIG. 1A, 1B) using a cryo-EM structure of E. coli type 1 pili as a guide (PDB: 6c53) (Hospenthal et al., Structure 25, 1829-1838.e1824 (2017), Spaulding et al., eLife 7, e31662 (2018)), and mutation-dependent 10- to 84-fold increases in the conductivity of the filaments were demonstrated. Importantly, the work utilizes methods to measure the conductivity of individual filaments rather than filament films, which is distinct to prior conductivity studies of protein networks where high contact resistance between filaments and measurement electrodes has masked the intrinsic electronic properties of the individual filaments (Kalyoncu et al., RSC Adv. 7, 32543-32551 (2017), Dorval Courchesne et al., Nanotechnology 29, 454002 (2018), Chen et al., Nature Materials 13, 515-523 (2014)). The approach also eliminates network artifacts where conductivity is dominated by percolation behavior, and reduces the impact of inter-filament contact resistance on the conductivity measurements (Shipps et al., Proceedings of the National Academy of Sciences 118, e2014139118 (2021)). Next, long-range conductivity over the micrometer scale was engineered by generating networks of hierarchical assemblies of conductive pili using molecular recognition self-assembly (Cao et al., Angewandte Chemie 50, 6264 (2011)) (FIG. 1C). Finally, a genomically recoded strain of E. coli (Lajoie et al., Science 342, 357-360 (2013)) was used to genetically encode the nonstandard amino acid (nsAA) propargyloxy-phenylalanine (PrOF) to generate pili that form a click-chemistry-functional scaffold for the precise, site-specific conjugation of gold nanoparticles (AuNPs), leading to the biosynthesis of sequence-controlled organic-inorganic hybrid biomaterials endowed with conductivity enhanced by 170-fold (FIG. 1D).

### Example 1: Engineered Biomaterial Platform

### Materials and Methods

### Bacterial Strains:

All protein expression was done in strains derived from E. coli C321.A fimBE::tolC (strain A), where fimBE genes were deleted using λ-red recombination by replacing them with a tolC cassette. E. coli C321.A fimA::gent (strain B) was created by deleting fimA with gentamycin using λ-red recombination. fimA mutants in the fimBE::tolC background were created using multiplex automated genome engineering (MAGE) (Gallagher et al., Nature Protocols 9, 2301-2316 (2014)). E. coli strain NEB5α was used for cloning and plasmid assembly (New England Biolabs).

### Growth Conditions:

When necessary, chloramphenicol, kanamycin, and gentamycin were used at 30, 20, and 5 μg/mL, respectively. SDS was used at 0.005% w/v for fimBE::tolC selection. All C321 cells were grown in Luria-Bertani broth containing 5 g/L NaCl supplemented with relevant antibiotics or SDS. NEB5α cells were recovered in SOC medium as described in the NEB5α competent cell protocol (New England Biolabs).

### Plasmids Used:

To create plasmid pSHDS.1, the fimA-fimH pili operon from the chromosome of genetically recoded organism E. coli C321.A (C321) was cloned out via PCR and Gibson assembly was then used to insert fimA-H into a plasmid based on pZE21G (Lajoie et al., Science 342, 357-360 (2013)) under control of an aTc-inducible promoter. Plasmids pSHDS.80 and pSHDS.109 were made by creating mutations fimA A80TAG or A109TAG in pSHDS.1, respectively, using the Q5 site-directed mutagenesis protocol (New England Biolabs). Plasmid pAzFRS.2.t1 (Amiram et al., Nature Biotechnology 33, 1272-1279 (2015)) (addgene: addgene.org/73546/) was used to express the orthogonal translation system (OTS) that allows for the incorporation of nonstandard amino acids (NSAAs) into proteins.

### Results

Type 1 pili are micrometre-long helical polymers composed of thousands of copies of the FimA protein (Lillington et al., Biochimica et Biophysica Acta (BBA)—General Subjects 1840, 2783-2793 (2014), Alonso-Caballero et al., Nature Communications 9, 2758 (2018), Hospenthal et al., Structure 25, 1829-1838.e1824 (2017), Spaulding et al., eLife 7, e31662 (2018), Sheikh et al., PLoS Negl Trop Dis 11, e0005586 (2017)) and can be purified in large quantities at high purity (FIG. 1A, right). E. coli was used as a chassis organism because it is easy to grow, easy to genetically manipulate, can incorporate nsAAs, and expresses type 1 pili naturally (FIG. 1A, left). The E. coli strain employed to express all type 1 pili variants was a previously described E. coli MG1655 derivative that facilitates accurate insertion of nsAAs into proteins with high efficiency (Lajoie et al., Science 342, 357-360 (2013), Amiram et al., Nature Biotechnology 33, 1272-1279 (2015), Isaacs et al., Science 333, 348-353 (2011)). This E. coli strain, known as the genomically recoded organism (GRO), has all instances of UAG stop codons re-coded to synonymous UAA codons and lacks release factor 1 (RF1), establishing the UAG codon as an open codon. The open UAG codon, together with an engineered orthogonal translation system (OTS) encoding an orthogonal M jannaschii tRNA-aminoacyl-tRNA synthetase pair, allows for the efficient and site-specific incorporation of diverse nsAAs (Lajoie et al., Science 342, 357-360 (2013), Amiram et al., Nature Biotechnology 33, 1272-1279 (2015)). Taken together, this recoded strain of E. coli permits the production of proteins with multiple instances of nsAAs at high yield and accuracy (Amiram et al., Nature Biotechnology 33, 1272-1279 (2015)), setting the stage for functionalizing pili biomaterials with synthetic chemical modalities.

### Example 2: Aromatic Amino Acid Mutations in Type 1 Pili Increase Conductivity on the Nanometre Scale

### Materials and Methods

### Multiplex Automated Genome Engineering (MAGE) and λ-Red Recombination:

MAGE and λ-RED recombination were conducted as described elsewhere (Gallagher et al., Nature Protocols 9, 2301-2316 (2014)). In short, liquid cultures were inoculated from frozen stock and grown overnight. These cultures were back-diluted 1:100 and grown to mid-logarithmic growth (OD₆₀₀˜0.6) in a shaking incubator at 34° C. λ-red recombination proteins Exo, Beta, and Gam were expressed by keeping the cells shaking in a water bath at 42° C. for 15 minutes. Cells were immediately chilled on ice and moved to a 4° C. environment. 1 mL of cells was centrifuged at 16000×g for 15s. The supernatant was removed, and the cells were resuspended in milli-Q water. The cells were again spun down, the supernatant was removed, and the cells resuspended in fresh milli-Q water to wash. This process was repeated three times. After the final spin, the supernatant was removed, and either mutagenic MAGE oligos prepared at 5-6 μM in DNase-free water or 50 ng of dsDNA were added directly to the cell pellet and mixed thoroughly. The oligo-cell mixture was applied to a pre-chilled 1 mm gap electroporation cuvette (Bio-Rad) and electroporated at 1.8 kV, 200 V and 25 mF. The cells were immediately resuspended in 2 mL LB broth and recovered at 34° C. in a shaking incubator for 4 hours. When the cells again reached mid-logarithmic growth additional MAGE cycles were conducted or the cells were plated for future analysis.

Successful incorporation of mutations in the fimA gene of C321 fimBE::tolC was screened using MASC-PCR as described elsewhere (Gallagher et al., Nature Protocols 9, 2301-2316 (2014)). dsDNA designed to replace fimBE with tolC and fimA with gentR contained the tolC or gentR cassettes with overhangs to regions directly before and after fimBE or fimA. Successful incorporation of tolC or gentR was confirmed by selection on SDS or gentamycin and sequencing of the fimBE and fimA regions.

### Oligonucleotides:

Oligonucleotides, including primers and MAGE oligos, were purchased from Keck Oligonucleotide Laboratory at Yale University.

### Chromosomal Pili Expression:

WT pili or those containing standard amino acid mutations (F, Y, or W) encoded genomically in strain A were expressed by inoculating three 500-mL flasks of LB from frozen glycerol stocks and growing the cultures at 34° C. without shaking. These cultures were grown in such conditions for 48 hours, after which cells were either imaged with TEM to visualize pili production, assayed for pili production using yeast agglutination, or used for pili protein harvesting and subsequent purification.

### Yeast Agglutination Assay for Pili Production:

100 μL of yeast grown overnight from frozen stock and diluted to an OD₆₀₀ of ˜1 in PBS pH 7.4 (−) MgCl₂ (−) CaCl₂) was mixed with 100 μL of E. coli grown in pili-producing conditions diluted to an OD₆₀₀ of ˜1 in PBS pH 7.4 (−) MgCl₂ (−) CaCl₂. The mixture was incubated at room temperature and shaken at 350 rpm for 15 min. 1.5 μL of mixture was pipetted onto a glass slide and the mixture imaged using a standard optical microscope. Visible agglutination of yeast cells indicated production of pili by E. coli cells. Agglutination was detected qualitatively, and pili production was confirmed with TEM imaging of E. coli samples which produced an agglutination phenotype. Adapted from Firon et al. (Firon et al., Infection and Immunity 55, 472-476 (1987))

### Pili Purification:

Cells grown in pili producing conditions were spun down in 4° C. at 6,000 rpm for 10 min and resuspended in 150 mM ethanolamine (ETA) pH 10.5. Cells were vortexed in a 50 mL conical tube for 2 min at vortex level 9 to shear pili from the cells, after which the cells were centrifuged in 4° C. at 10,000 rpm for 45 minutes. The supernatant was kept, and the cell pellet was discarded. The supernatant was then centrifuged in 4° C. at 23,000×g for 1 h to remove remaining contaminants. The supernatant of this spin was kept, and the debris pellet was discarded. Saturated ammonium sulfate (SAS) (purchased from Thermo Fisher) was added to the supernatant to 15% of the final volume to precipitate out pili proteins. The solution was left static at 4° C. overnight, and then spun in 4° C. at 23,000×g for 1 h to collect precipitated pili. The supernatant was discarded, and the pili pellet was resuspended in ˜600 μL 150 mM ETA pH 10.5. To this sample, 1 M MgCl₂ was added to a final concentration of 100 mM MgCl₂ and incubated at 4° C. overnight to precipitate out pili. The solution was then spun in 4° C. at 17,000×g for 1 h to pellet pili. The supernatant was discarded, and the pili pellet was resuspended and solubilized in 150 mM ETA pH 10.5 by gentle pipetting to a volume of 150-500 μL depending on pellet size. The pili sample was then imaged or used for conductivity measurements. To determine purity of the sample via SDS-PAGE, the following steps were followed. To the pili sample solubilized in 150 mM ETA pH 10.5 in the previous step crystalline urea was added until the concentration was 6M and the solution was then kept at room temperature for 4 hours. The solution was then eluted through a 20 mL Sepharose column using 6M urea as the eluent. The first 5 mL was taken as the void volume, followed by 15 fractions, each being 1 mL in volume. Fraction 2 was found to contain the purified pili filaments. Fraction 2 was then concentrated to 60 μL using a 3 kDa-cutoff Amicon Ultra-0.5 mL Centrifugal Filter. 15 μL of the concentrated fraction 2 sample was used for further gel analysis as follows. To the 15 μL sample 5 μL of 4×Laemmelli buffer with 5% Beta-mercaptoethanol (BioRad) was added. The sample was then boiled for 25 minutes and then run on a SYPRO Ruby-stained SDS-PAGE gel. Once the presence and purity of FimA was confirmed, the remaining 45 μL volume of concentrated fraction 2 was then increased to 250 μL with 6M Urea and subsequently dialyzed into 150 mM ETA pH 10.5. This protocol is partially inspired by a similar purification methodology (Korhonen et al., Infection and Immunity 27, 569-575 (1980)).

### Transmission Electron Microscopy (TEM) Imaging of Pili Samples:

Carbon film copper grids with mesh size 400 (Electron Microscopy Sciences) were cleaned with a PlasmaFlow plasma cleaner on medium for 30 s. 5 μL of pili sample was then dropcast onto the copper grid and left to adhere to the grid for 10 min, after which the remaining buffer was blotted off using filter paper. The samples were stained with a 1% PTA stain pH 6 by floating the grids on 50 μL droplets of stain for 30 s, removing the grid and blotting the stain off with filter paper, floating the grids on the stain a second time for 30 s, and finally removing the grids and blotting the stain off with filter paper. The grids were then air dried for 10 min before storage and imaging.

### Fluorescence Measurements:

A time-based assay was conducted on a Cary 3E UV-Vis Spectrophotometer with excitation wavelengths at 280 nm and 295 nm. The excitation bandwidth was 2.5 nm, with the emission bandwidth at 10 nm. The emission scan range for the 280 nm excitation was 295 nm to 500 nm and the emission scan range for the 295 nm excitation was 310 nm to 500 nm. The step size for the scan was 1 nm. The scan rate was 100 nm/min. The sample used was 125 μL of FimA A80 W A109 W pili and 125 μL FimA WT pili, dissolved in milli-Q water (pH 7.0) or 150 mM ETA pH 10.5. The emission intensity at each wavelength in the scan range was measured in counts per second. The background emission of the solvent at 125 μL was collected and subtracted to yield the final results.

### Electrode Device Sample Preparation:

Electrode devices were washed with acetone, isopropanol, ethanol, and Milli-Q water, in that order, two times. After the second water wash, the device was left to air dry. The device was then plasma cleaned with the electrode side facing up using a tabletop Harrick Plasma cleaner on low for 2 min. The electrode was taken out of the plasma cleaner, and 3 μL of pili sample was immediately dropcast onto the centre of the electrode, which was then left to dry in a desiccator at 20% humidity overnight. The buffer was removed by washing the electrode surface with 17 μL Milli-Q water three times. After the third wash, the electrode was left to air dry for at least 45 min. Electrodes bridged by E. coli pili were located using (Asylum Research).

### Atomic Force Microscopy (AFM) Imaging:

Soft cantilevers (AC240TS-R3, Asylum Research) with a nominal force constant of 2 N/m and resonance frequencies of 70 kHz were used. The free-air amplitude of the tip was calibrated with the Asylum Research software, and the spring constant was captured by the thermal vibration method. The sample was imaged with a Cypher ES scanner using intermittent tapping (AC-air topography) mode. Images were analysed using Asylum research v.16 and Gwyddion v.2.55.

### Pili Conductance Measurements for Individual Filaments:

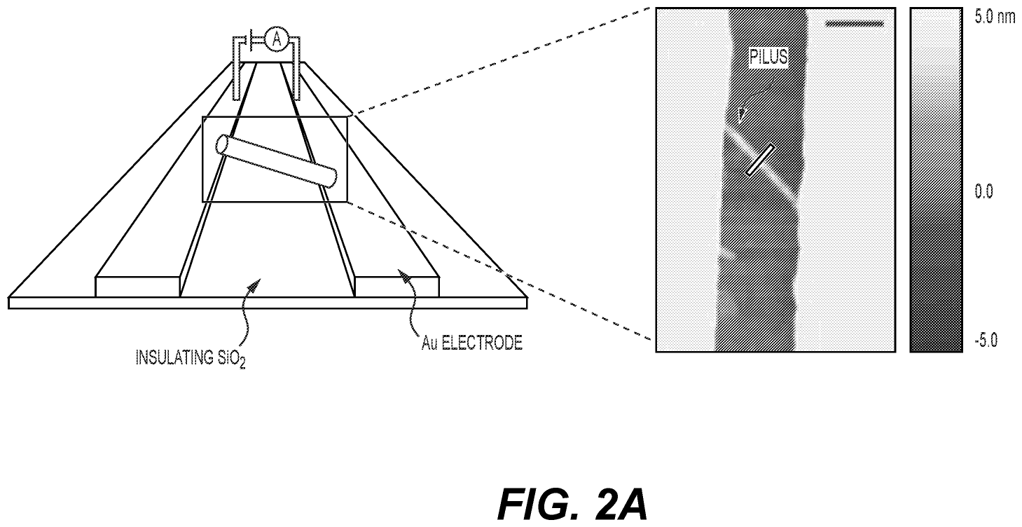

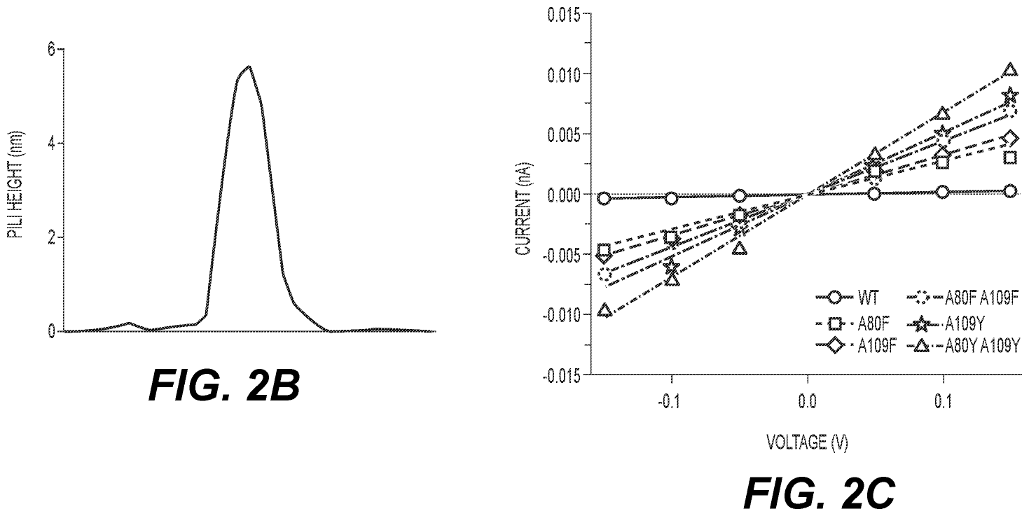

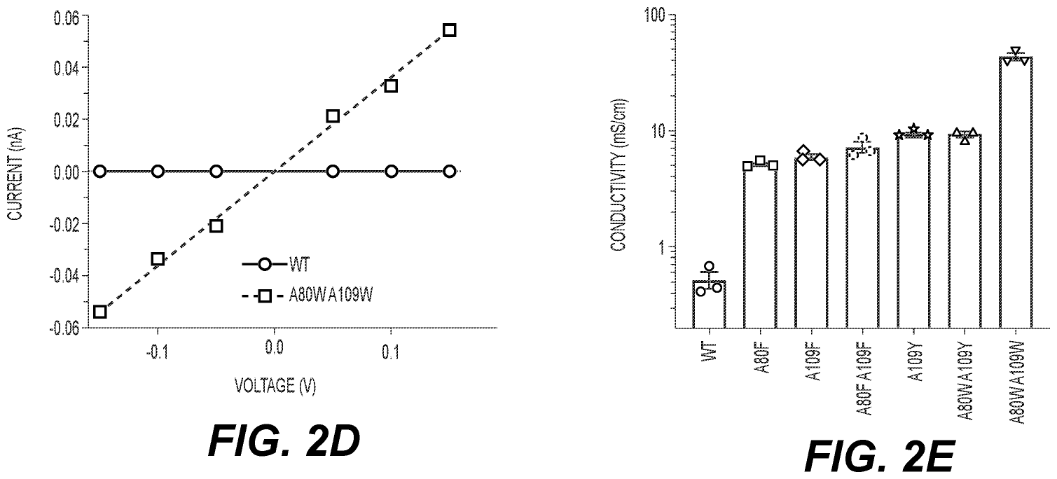

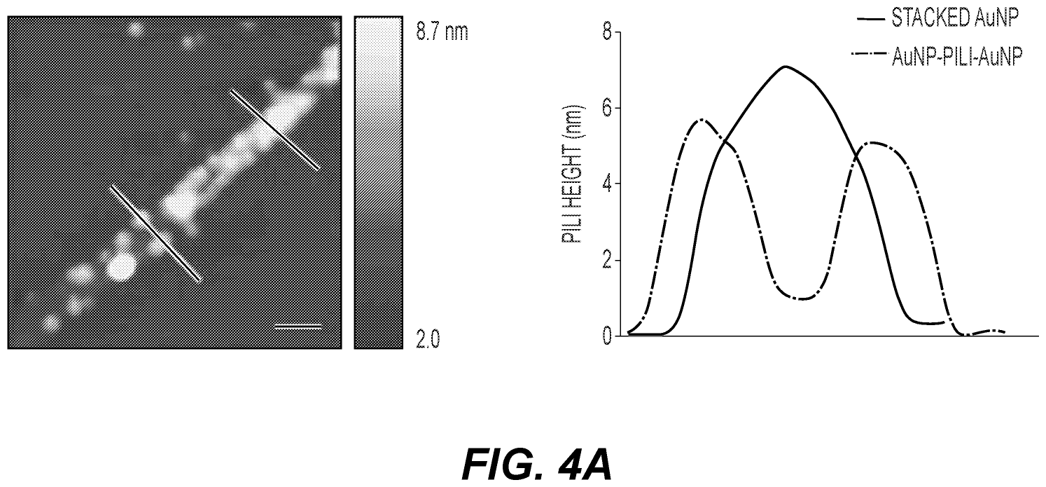

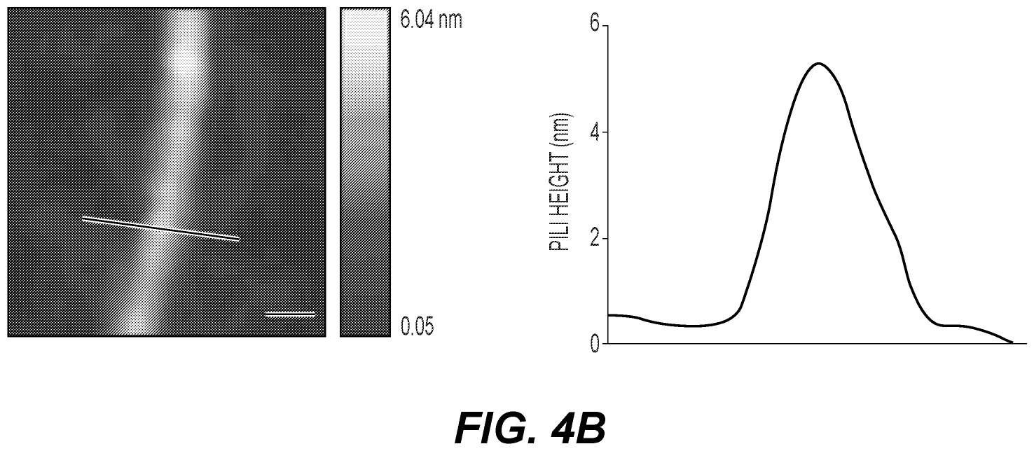

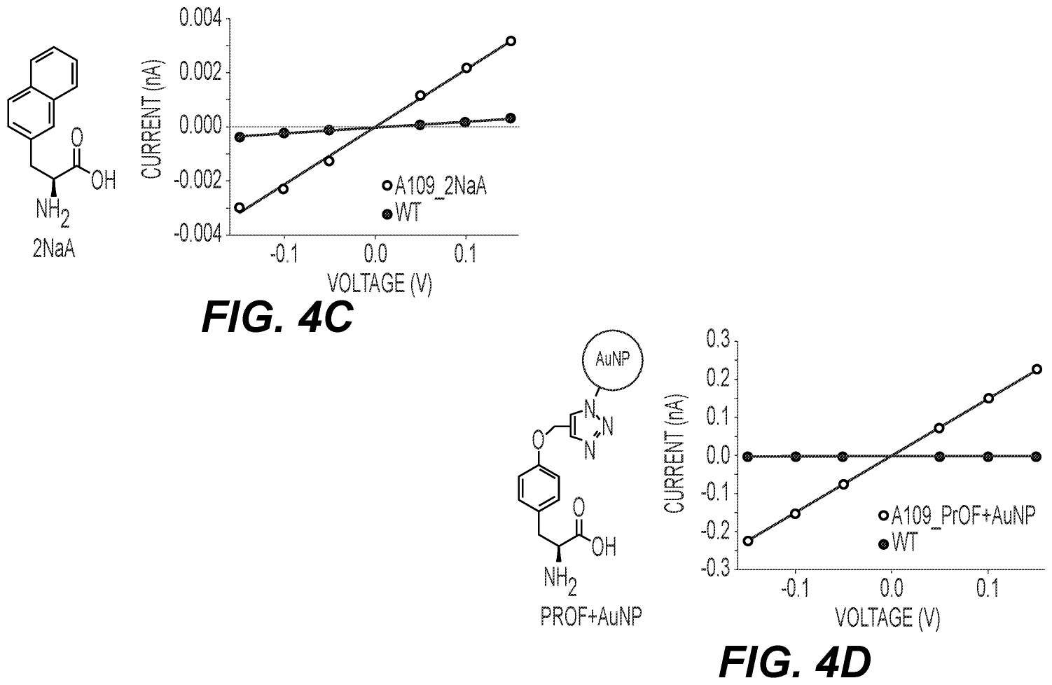

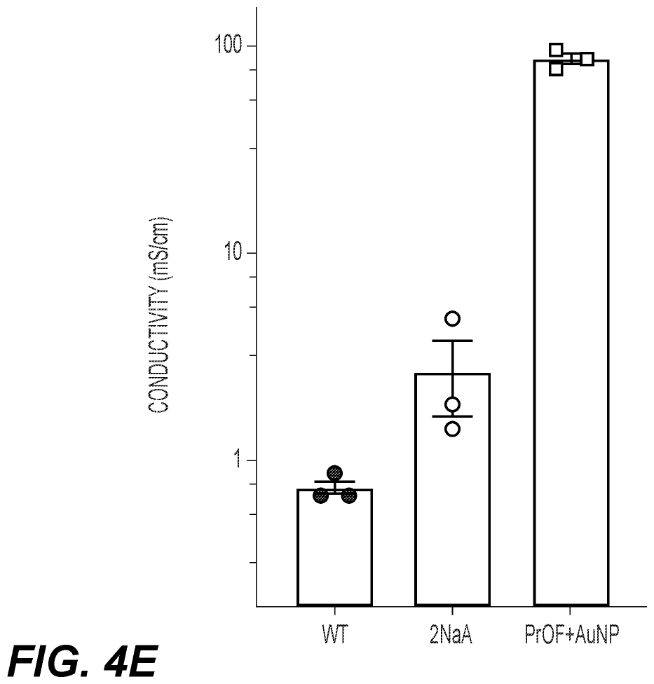

Conductance measurements were performed as described previously (Wang et al., Cell 177, 361-369.e310 (2019)). Electrode devices with 17 electrodes spaced 300 nm apart were imaged under AFM to find individual pili filaments bridging two electrodes. Devices with pili bridging two electrodes were re-hydrated by dropping 0.3 μL of 150 mM ETA pH 10.5 onto the electrode and waiting 45 minutes. Conductance G of pili was measured in a 2-electrode configuration inside a shielded dark box using an MPI Corporation probe station connected to a semiconductor parameter analyser (Keithley 4200A-SCS). DC voltages from −0.15 to 0.15 V in increments of 0.05 V were applied between electrodes bridged by pili and current A was measured until a steady state was observed, typically over a period of two minutes. The linearity of the I-V characteristics was maintained by applying an appropriate low voltage and the slope of the I-V curve was used to determine the conductance (G). IV linear fits did not always go through (0,0), however conductivity was derived from the slope of the IV linear fit. Measurements were performed at low voltages (<0.15 V) and over longer times (>120 s) to ensure a lack of electrochemical leakage currents or faradic currents as evidenced by the absence of significant DC conductivity in buffer. All analysis was performed using IGOR Pro v.7 (WaveMetrics Inc.). Data was graphed using Graphpad Prism v.8. To display representative current-voltage curves in FIGS. 2 and 4, the y-intercept of the raw linear fit was subtracted from the value of each data point such that all linear fits for the data had a y-intercept of zero. This display method preserves the slope of the linear fit and thus the conductance measurement of the filament, and allows for easy comparison of the differences in the magnitude of the conductance between filaments. Raw current-voltage data for all filaments is reported in Supplementary FIG. 3 and the source data file.

### Pili Conductivity Calculations for Individual Filaments:

The conductivity σ of filaments was calculated using the relation described elsewhere (Malvankar et al., Nature Nanotechnology 6, 573-579 (2011))

$$σ = \frac{GL}{na}$$

where G is conductance, L is length of the filament measured between the electrodes, a=πr² is area of cross section of the filament with 2r as the height of the filament as measured by AFM, and n is number of filaments bridging the electrodes in series. For pili with gold nanoparticles attached, the height was measured as pili+AuNP, as in each case the gold nanoparticles completely covered the part of the filament between electrodes. All analysis was performed using IGOR Pro v.7 (WaveMetrics Inc.) Data was graphed using Graphpad Prism v.8.

### DNA Sequences

WT fimA Sequence of E. coli C321.A.

The mutation locations are based on the sequence without the signal peptide (highlighted in bold and italics). Locations A80, H82, and A109 (after cleavage of the signal peptide) are highlighted in dotted underline, dashed and dotted underline, and solid underline, respectively.

(SEQ ID NO: 3)
atgAAAATTAAAACTCTGGCAATCGTTGTTCTGTCGGCTCTGTCCCTCAGT
TCTACAGCGGCTCTGGCCGCTGCCACGACGGTTAATGGTGGGACCGTTCAC
TTTAAAGGGGAAGTTGTTAACGCCGCTTGCGCAGITGATGCAGGCTCTGTT
GATCAAACCGTTCAGTTAGGACAGGTTCGTACCGCATCGCTGGCACAGGAA
GGAGCAACCAGTTCTGCTGTCGGTTTTAACATTCAGCTGAATGATTGCGAT
ACCAATGTTGCATCTAAAGCCGCTGTTGCCTTTTTAGGTACGGCGATTGAT [symbol]
GGT [symbol] ACCAACGTTCTGGCTCTGCAGAGTTCAGCTGCGGGTAGCGCA
ACAAACGTTGGTGTGCAGATCCTGGACAGAACGGGTGCTGCGCTGACGCTG
GATGGTGCGACATTTAGITCAGAAACAACCCTGAATAACGGAACCAATACC
ATTCCGTTCCAGGCGCGTTATTTTGCAACCGGGGCCGCAACCCCGGGTGCT
GCTAATGCGGATGCGACCTTCAAGGTTCAGTATCAATAA

### MAGE Oligonucleotides

A table of all oligonucleotides used to create chromosomal mutations in the fimA gene. The mutations are highlighted in bold. An asterisk * denotes a phosphorothioate bond.

TABLE 2
Oligonucleotides used to create chromosomal mutations

| Mutation | |
|---|---|
| in fimA | Oligonucleotide sequence with mutation highlighted. |
| A109F | A*T*TCAGGGTTGTTTCTGAACTAAATGTCGCACCATCCAGCGTCAGCGCGAAA |
| | CCCGTTCTGTCCAGGATCTGCACACCAACGTTTGTTGC (SEQ ID NO: 4) |
| A80F | A*G*CTGAACTCTGCAGAGCCAGAACGTTGGTATGACCGAAATCAATCGCCGTA |
| | CCTAAAAAGGCAACAGCGGCTTTAGATGCAACATTGGT (SEQ ID NO: 5) |
| H82F | A*G*CTGAACTCTGCAGAGCCAGAACGTTGGTGAAACCCGCATCAATCGCCGTA |
| | CCTAAAAAGGCAACAGCGGCTTTAGATGCAACATTGGT (SEQ ID NO: 6) |
| A109Y | A*T*TCAGGGTTGTTTCTGAACTAAATGTCGCACCATCCAGCGTCAGCGCATAA |
| | CCCGTTCTGTCCAGGATCTGCACACCAACGTTTGTTGC (SEQ ID NO: 7) |
| A80Y | A*G*CTGAACTCTGCAGAGCCAGAACGTTGGTATGACCATAATCAATCGCCGT |
| | ACCTAAAAAGGCAACAGCGGCTTTAGATGCAACATTGGT (SEQ ID NO: 8) |
| H82Y | A*G*CTGAACTCTGCAGAGCCAGAACGTTGGTATAACCCGCATCAATCGCCGTA |
| | CCTAAAAAGGCAACAGCGGCTTTAGATGCAACATTGGT (SEQ ID NO: 9) |
| A80F | A*G*CTGAACTCTGCAGAGCCAGAACGTTGGTGAAACCGAAATCAATCGCCGTAC |
| H82F | CTAAAAAGGCAACAGCGGCTTTAGATGCAACATTGGT (SEQ ID NO: 10) |
| A80Y | A*G*CTGAACTCTGCAGAGCCAGAACGTTGGTATAACCATAATCAATCGCCGTA |
| H82Y | CCTAAAAAGGCAACAGCGGCTTTAGATGCAACATTGGT (SEQ ID NO: 11) |
| A80F | A*G*CTGAACTCTGCAGAGCCAGAACGTTGGTGAAACCATAATCAATCGCCGT |
| H82Y | ACCTAAAAAGGCAACAGCGGCTTTAGATGCAACATTGGT (SEQ ID NO: 12) |
| A80Y | A*G*CTGAACTCTGCAGAGCCAGAACGTTGGTATAACCGAAATCAATCGCCGT |
| H82F | ACCTAAAAAGGCAACAGCGGCTTTAGATGCAACATTGGT (SEQ ID NO: 13) |
| A109W | A*T*TCAGGGTTGTTTCTGAACTAAATGTCGCACCATCCAGCGTCAGCGCCCA |
| | ACCCGTTCTGTCCAGGATCTGCACACCAACGTTTGTTGC (SEQ ID NO: 14) |
| A80W | A*C*CCGCAGCTGAACTCTGCAGAGCCAGAACGTTGGTATGACCCCAATCAATC |
| | GCCGTACCTAAAAAGGCAACAGCGGCTTTAGATGCAAC (SEQ ID NO: 15) |
| A80F | G*C*GCTACCCGCAGCTGAACTCTGCAGAGCCAGAACGAAGGTATGACCGAAAT |
| A84F | CAATCGCCGTACCTAAAAAGGCAACAGCGGCTTTAGAT (SEQ ID NO: 16) |
| A80Y | A*G*CTGAACTCTGCAGAGCCAGAACATAGGTATGACCATAATCAATCGCCGT |
| A84Y | ACCTAAAAAGGCAACAGCGGCTTTAGATGCAACATTGGT (SEQ ID NO: 17) |
| A84F | G*C*GCTACCCGCAGCTGAACTCTGCAGAGCCAGAACGAAGGTATGACCCGCA |
| | TCAATCGCCGTACCTAAAAAGGCAACAGCGGCTTTAGAT (SEQ ID NO: 18) |
| A84Y | G*C*GCTACCCGCAGCTGAACTCTGCAGAGCCAGAACATAGGTATGACCCGCAT |
| | CAATCGCCGTACCTAAAAAGGCAACAGCGGCTTTAGAT (SEQ ID NO: 19) |

### Site-Directed Mutagenesis Primers:

Mutations FimA A80TAG and A109TAG were created using site-directed mutagenesis on the pSHDS.1 plasmid as in methods. Forward (F) and reverse (R) primers are listed below for each mutation.

| | |
|---|---|
| fimA A80TAG F | gggtCATACCAACGTTCTGGCTC |
| | (SEQ ID NO: 20) |
| fimA A80TAG R | taatcAATCGCCGTACCTAAAAAG |
| | (SEQ ID NO: 21) |
| fimA A109TAG F | ggcgCTGACGCTGGATGGTGCG |
| | (SEQ ID NO: 22) |
| fimA A109TAG R | taaccCGTTCTGTCCAGGATCTGC |
| | (SEQ ID NO: 23) |

### Results

E. coli pili with enhanced conductivity were engineered by generating a channel of aromatic amino acids along the filament. Recent studies have shown that the close stacking of aromatic tyrosine residues allows electron transfer in individual protein crystals over micrometre-long distances (Shipps et al., Proceedings of the National Academy of Sciences 118, e2014139118 (2021)). These studies contrast the widely held belief that proteins are considered to be electronic insulators that can only transfer electrons over a few nanometres (Zhang et al., Proceedings of the National Academy of Sciences 116, 5886-5891 (2019)). Indeed, several non-aromatic proteins have recently been shown to be conductive over nanometres in single-molecule measurements, with little decay due to distance, provided charge is precisely injected into the protein interior with good contact. However, the conduction mechanism is unknown (Zhang et al., Journal of the American Chemical Society 142, 6432-6438 (2020)). As type 1 pili are common virulence factors in some pathogenic strains of E. coli, it has been observed that many residues on the outside of type 1 pili are highly variable to avoid immune response (Sheikh et al., PLoS Negl Trop Dis 11, e0005586 (2017)). Thus, a strategy was developed to identify external residues that would permit mutations without altering their structure substantially. Candidate residues that were both in the set of variable surface residues and which, when mutated, would be in close enough proximity (<15 Å) to each other to facilitate efficient electron transfer, likely through electron hopping, along the pilus: FimA A80, H82, and A109 (FIG. 1B).

Two assays were performed to confirm the identity of the purified pili encoded by FimA. First, SDS-PAGE gel electrophoresis was used to confirm that the only purified protein in the samples was FimA. Second, an established yeast agglutination assay was used in which Saccharomyces cerevisiae cells agglutinate in the presence of E. coli expressing type 1 pili but do not when mixed with E. coli that do not express type 1 pili (Sheikh et al., PLoS Negl Trop Dis 11, e0005586 (2017), Korhonen, FEMS Microbiology Letters 6, 421-425 (1979)). Yeast agglutination behavior is dependent on E. coli pili expression, either from the genome or from a plasmid as used in this study. Deleting fimA from the genome abolishes yeast agglutination, while expressing fimA from a plasmid rescues yeast agglutination.

A multiplex automated genome engineering (Gallagher et al., Nature Protocols 9, 2301-2316 (2014), Wang et al., Nature 460, 894-898 (2009)) (MAGE, see methods) was next used to generate a library of FimA mutants in which the native amino acids A80, H82, or A109 were replaced with the aromatic amino acids phenylalanine, tyrosine, histidine, and tryptophan (Table 3) and the impact of these mutations on FimA pili expression was assessed using Transmission Electron Microscopy (TEM).

TABLE 3
Standard Amino Acid mutations made in type 1 pili

| | Pili | |
|---|---|---|
| Mutation in fimA | Expression | σ (mS/cm) |
| WT | Yes | 0.5198 ± 0.1475 |
| A80F | Yes | 5.168 ± 0.2660 |
| A109F | Yes | 5.933 ± 0.5713 |
| A80F A109F | Yes | 7.250 ± 1.355 |
| A80F H82F A109F | Not detected | |
| A80F H82F A109Y | Not detected | |
| A80Y | Yes | 9.208 ± 0.6528 |
| A109Y | Yes | Not Measured |
| A80Y A109Y | Yes | 9.287 ± 0.872 |
| A80W | Yes | Not Measured |
| A109W | Yes | Not Measured |
| A80W A109W | Yes | 43.48 ± 4.572 |
| A80Y H82F A80Y | Not detected | |
| A80Y H82Y A109Y | Not detected | |

Eight mutations in FimA were found that preserve pili expression and assembly and the conductivity of six mutants: A80F, A109F, A80F A109F (double mutant), A109Y, A80Y A109Y (double mutant), and A80 W A109 W (double mutant) were measured (FIG. 1A, Table 3). Four pili variants were made with mutants at position 82, and all resulted in the loss of pili expression (Table 3). It was believed that mutant pili with two additional aromatic residues per monomer would be more conductive than those with only one additional aromatic amino acid per monomer, as the aromatic groups would be in closer proximity to one another and facilitate enhanced electron transport along the pilus.

To determine the conductivity of individual pili filaments, pili were placed on gold electrodes separated by 300 nm nonconductive gaps and located individual filaments bridging two electrodes using atomic force microscopy (AFM, FIGS. 2A, 2B). Voltages ranging from −0.15V to +0.15V were applied across the filament and measured the steady-state current through the pili. After measuring the conductance as the slope of the current-voltage curve, the conductivity was calculated as reported previously (Malvankar et al., Nature Nanotechnology 6, 573-579 (2011)). Wild-type pili filaments showed very low conductivity (FIG. 2A-2E) of 0.51±0.14 mS/cm. Incorporating a single phenylalanine mutation (A80F or A109F) in each FimA monomer increased the conductivity of individual pili filaments by 10-fold to 5.168±0.154 mS/cm for A80F and 5.933±0.323 mS/cm for A109F. Incorporating a single tyrosine mutation (A109Y) in each FimA monomer increased the conductivity of individual pili filaments 18-fold to 9.208±0.377 mS/cm (FIG. 2C, 2E). Incorporating two phenylalanine residues per monomer (A80F A109F) generated pili that were slightly more conductive than the single phenylalanine mutants at 7.250±0.782 mS/cm, but still less conductive than pili incorporating tyrosine. Pili incorporating two tyrosine residues per monomer (A80Y A109Y) were only slightly more conductive than those with one tyrosine per monomer at 9.287±0.504 mS/cm.

Next, pili with two tryptophan residues introduced per FimA monomer were used to study the associated conductivity of this engineered pili variant. Remarkably, incorporating two tryptophan mutations per FimA monomer (FimA A80 W A109 W) increased conductivity 84-fold to 43.48±4.53 mS/cm (FIG. 2D-2E) from wild-type. These results demonstrate an increase in the conductivity of individual pili filaments in the absence of metal co-factors, close amino acid packing, or high potentials typically required to achieve such conductivity in proteins (Kalyoncu et al., RSC Adv. 7, 32543-32551 (2017), Dorval Courchesne et al., Nanotechnology 29, 454002 (2018), Guterman & Gazit, Bioelectron Med (Lond) 1, 131-137 (2018), Taheri et al., Sci Rep 8, 9333 (2018)). This is believed to be the highest reported conductivity to-date of a single protein-based filament without metal cofactors with known atomic structure. Notably, the measured conductivity is comparable to the reported conductivity of metal-containing G. sulfurreducens filaments, comprised of cytochrome OmcS (51±11 mS/cm (Wang et al., Cell 177, 361-369.e310 (2019), Adhikari et al., RSC Adv. 6, 8354-8357 (2016))). Such remarkably high conductivity is likely due to highly π-stacked molecular structures owing to the indole side chain of tryptophan. In contrast to tyrosine, which requires protein environments that favor proton transfer, tryptophan residues can relay electrons at biologically relevant potentials even in protein environments that disfavour proton transfer (Shipps et al., Proceedings of the National Academy of Sciences 118, e2014139118 (2021), Shih et al., Science 320, 1760-1762 (2008)). Fluorescence spectroscopy confirmed that tryptophan substitution did not cause significant structural change in pili as the mutated tryptophan remained solvent-exposed in a manner similar to alanine residues in native pili structure. Therefore, these results demonstrate that the incorporation of two tryptophan residues per FimA monomer can increase the conductivity of individual pili filaments up to 84-fold in the absence of metal cofactors, close amino acid packing, or high potentials typically required to achieve such conductivity in proteins (Kalyoncu et al., RSC Adv. 7, 32543-32551 (2017), Dorval Courchesne et al., Nanotechnology 29, 454002 (2018), Guterman & Gazit, Bioelectron Med (Lond) 1, 131-137 (2018), Taheri et al., Sci Rep 8, 9333 (2018)).

### Example 3: Computationally-Guided Design of Hierarchical Nanostructures at the Micrometre Scale

### Materials and Methods

### Mica Sample Preparation:

A small square of mica (Electron Microscopy Sciences inc.) was attached to tape and the top layer was peeled off, leaving an atomically flat fresh layer of mica. Onto this layer of mica, 3-4 μL of pili solution was dropcast and left to dry in a desiccator at 20% humidity overnight. The buffer was removed by washing the electrode surface with 17 μL Milli-Q water. Washing was done by dropping 17 μL of water onto the mica surface, gently pipetting the bubble of water up and down a few times, pipetting off the water, and discarding the dirty water and tip. This process was repeated three times. After the third wash, the mica sample was left to air dry for at least 45 min, after which pili were imaged using an Asylum Cypher ES atomic force microscope (Asylum Research).

### Pili Bundling by Hexamethylenediamine:

Three different concentrations of hexamethylenediamine, 0 M, 0.08 M, and 0.25 M, were mixed with pure samples of FimA A80 W A109 W pili in 150 mM ETA at pH 10.5. At this pH, both amines of the hexamethylenediamine molecule are still protonated (Hébert et al., Toxicol. Sci. 20, 348-359 (1993)). These values were chosen based on the concentrations of hexamethylenediamine used elsewhere (Cao et al., Angewandte Chemie 50, 6264 (2011)). One original sample of FimA A80 W A109 W pili was divided among the three hexamethylene concentrations to normalize pili concentration between samples. These solutions were stored at 4° C. for 7 days in order to allow bundles to form. Afterwards, the solutions were imaged using AFM to determine if bundles were present. 0 M hexamethylenediamine was used as the negative control for no bundling, 0.08 M was found to be too small for bundling in WT pili, and 0.25 M was found to cause bundling in WT pili.

### Pili Conductance Measurements for Bundles:

An interdigitated electrode (IDE) with a 5 Vm spacing between the finger electrodes was cleaned with acetone and dried using nitrogen gas. 0.5 μL of the FimA A80 W A109 W pili sample was drop cast onto the IDE and the sample was placed in a desiccator for 30 minutes to dry. Then, 5 μL of milli-Q water was added to the area where the pili were dropcast. Water was left on the sample for 1 minute, after which it was wicked away with filter paper. The sample was placed in the probe station, and the current was measured between −0.2 and 0.2 V in increments of 0.05 V over 150 s using the same setup as for measuring individual filaments. The conductance G of the pili network was found using the same method as for individual filaments.

### Pili Conductivity Calculations for Bundles:

Conductivity was calculated by using the thin-film measurement formula described elsewhere (Malvankar et al., Nature Nanotechnology 6, 573-579 (2011)),

$$σ = \frac{GL}{A} ,$$

where G is the conductance of the film, L is the length of the gap of the electrode, 5 microns, and A is the size of the drop that covers the electrode, which, after measurement, was estimated to be 0.926 μm².

### MD Simulation of Pili Bundling:

The atomic structure of the FimA monomer was obtained from the protein data bank (PDB ID 6C53). Polymerization of FimA into the type I pilus involves the insertion of an N-terminal beta strand formed by the first 20 residues of the neighboring subunit into an open groove in the immunoglobulin-like fold formed by residues 20 to 158 of the subunit of interest (Spaulding, et al., eLife 7, e31662 (2018)). To construct the simulation model, the first 18 residues were truncated and residues 3 to 20 of the neighboring subunit were added resulting in a complete immunoglobulin-like fold without an extraneous beta strand. Two of these monomer subunits were separated by a distance of 40 Å and rotated such that their outer surfaces faced each other. The hexamethylenediamine (HMD) molecule was parameterized using the CHARMM-GUI (Jo et al., Journal of Computational Chemistry 29, 1859-1865 (2008)) and the CHARMM (Brooks et al., Journal of Computational Chemistry 4, 187-217 (1983)) general force field in its fully protonated state with a charge of +2. For the simulation with 250 mM HMD, 55 HMD molecules were added around the protein, calculated as the number of HMD molecules required to solvate the box with 250 mM. Simulation systems both with and without HMD were solvated in a TIP3P water box with dimensions of 101×88×61 Å using the VMD (Humphrey et al., Journal of Molecular Graphics 14, 33-38 (1996)) solvate plugin. This provides at least 12 Å between the edge of the box and the closest protein atom. The Particle-Mesh Ewald (PME) method (Essmann et al., J. Chem. Phys. 103, 8577-8593 (1995)) was utilized to calculate long-range electrostatic interactions with 90 grid points in the x-direction, 80 grid points in the y-direction, and 54 grid points in the z-direction with a 12 Å cut-off. This spherical cut-off also extends to Lennard-Jones parameters. The temperature was maintained using a Langevin thermostat, and the pressure was maintained by using a constant Nosé-Hoover method in which Langevin dynamics is used to control fluctuations in the barostat. The velocity Verlet algorithm was used with a time-step of 1 fs.

MD simulations were performed with NAMD v2.13 (Phillips et al., Journal of Computational Chemistry 26, 1781-1802 (2005)) using the CHARMM36 (Best et al., Journal of Chemical Theory and Computation 8, 3257-3273 (2012)) force-field parameters with periodic boundary conditions. First, each system was minimized, followed by a 500 ps simulation of the water box and any HMD molecules with fixed protein atoms. The models were then equilibrated to 310 K in the NVT ensemble for 3.5 ns under harmonic restraints with a force constant of 0.1 kcal/mol to the amino acid sidechains and a force constant of 1.0 kcal/mol to the protein backbone. Production runs were then performed in an NPT ensemble for 100 ns with frames being written to the trajectory every 2.5 ps. Analysis was performed by computing the distance between the geometric centres of each monomer subunit using a custom Tcl script available as part of the supplementary information. All of the simulations were done on the Grace supercomputing cluster, a computational core facility located at Yale University's Centre for Research Computing.

### Results

Next, a strategy was designed to build on the development of the mutant filaments with increased conductivity at the nanometre scale by constructing pili-based nanostructures that are conductive at the micrometre scale (FIG. 1C). This effort is inspired by living systems, which create functional materials through hierarchical self-assembly of nanoscale molecules. Although synthetic molecules can be assembled into artificial nanostructures, bridging from the nanoscale to the macroscale to create functional macroscopic materials remains a challenge (Knowles et al., Nat. Nanotechnol. 5, 204-207 (2010)). The strategy for creating filament nanostructures aimed to use the molecule hexamethylenediamine (HMD) to align conductive pili into bundled lattices through molecular recognition-based self-assembly (Cao et al., Angewandte Chemie 50, 6264 (2011)) (FIG. 3A). Previous (Cao et al., Angewandte Chemie 50, 6264 (2011)) demonstrated the ability to create different nanostructures by modulating the identity and concentration of inducer. In this study, one inducer, HMD, was used to create 2D bundled lattices, to investigate the effect of self-assembly on pili material conductivity. HMD is positively charged at both ends and thus is able to promote alignment of negatively-charged pili (FIG. 3A) into larger structures. To evaluate the use of HMD in the formation of ordered nanowire assemblies, molecular dynamics simulations were performed on two monomers of the FimA A80 W A109 W pili with and without HMD. Results show that over the course of a 100 ns simulation, orientations of the pilin monomers that were aligned in close proximity with one another were only realized in the presence of 250 mM HMD (FIGS. 3B-3D), consistent with the conclusion that HMD could promote association of the filaments. As the MD simulations at 250 mM HMD set the number of HMD molecules to 55 (see: methods), two pilin monomers would set the protein concentration to be approximately 9.1 mM. Although this is higher than the protein concentration used experimentally, AFM images of pili on mica confirmed the MD-predicted ordering of FimA A80 W A109 W pili into one-layer bundles in the presence of 250 mM HMD (FIG. 3E).

To evaluate the conductivity of pili nanostructures at the micrometre scale, unordered networks of the FimA A80 W A109 W mutant, normalized for protein concentration, were placed on interdigitated electrodes with 5 m non-conductive gaps between each pair of electrodes. The conductance was measured and the conductivity calculated as described in methods. Conductance of HMD only shows a high baseline conductance of 0.3737±0.0472 nS, however conductance of unordered networks of FimA A80 W A109 W pili remains higher than background HMD at 0.5297±0.0113 nS. The conductance of bundled filaments is still significantly higher at 1.950±0.2495 nS. A Student's t-test was performed between ordered (A80 W A109 W pili, 250 mM HMD) and unordered (A80 W A109 W pili, 0 mM HMD), and the p-value=0.00007. It was also done between ordered and the control (250 mM HMD) and the p-value=0.0002. All experiments were performed independently, and error bars represent s.e.m. HMD only: n=3. Unordered pili: n=8. Bundled pili: n=11.

These pili networks show 100-fold higher conductivity than just HMD in the absence of pili at the micrometre scale, demonstrating remarkable long-range electron transfer (FIG. 3F). The conductivity of FimA A80 W A109 W nanostructures was then measure and it was found that assembling the pili networks into bundled nanostructures further increased conductivity by 5-fold, from 0.109±0.0023 S/cm to 0.535±0.06 S/cm (FIG. 3F). The measured conductivity of bundled FimA A80 W A109 W nanostructures is comparable to previous measurements of conductive microbial nanowire networks (Malvankar et al., Nature Nanotechnology 6, 573-579 (2011)). It is important to note that the conductivity of individual filaments and the conductivity of bulk films is not comparable, and that film conductivity is always lower than the conductivity of individual pili due to high contact resistance either between pili or between pili and electrodes. These results are consistent with previous studies on Geobacter protein nanowires that show film conductivity of 6 S/cm (Malvankar et al., Nature Nanotechnology 6, 573-579 (2011)) while individual filament conductivity is 0.5 mS/cm (Wang et al., Cell 177, 361-369.e310 (2019), Adhikari et al., RSC Adv. 6, 8354-8357 (2016)). Notably, other networks of conductive proteins comprised of engineered curli fibres have shown a significantly lower film conductivity than these pili (Dorval Courchesne et al., Nanotechnology 29, 454002 (2018), Creasey et al., ACS Omega 4, 1748-1756 (2019)). Although other self-assembly structures have been constructed by using different proteins, their conductivity has not been measured (Wu et al., Nature 574, 658-662 (2019)).

### Example 4: Sequence-Controlled Synthesis of Hybrid Organic-Inorganic Pili Increase Conductivity ˜170-Fold

### Materials and Methods

Nonstandard amino acid incorporation into pili and Subsequent Expression:

Pili with inserted 4-propargyloxy-L-phenylalanine (PrOF) as a fimA A109PrOF mutation were expressed as follows. Strain B was transformed with both plasmid pSHDS.109 and pAzFRS.2.t1 and inoculated into LB containing chloramphenicol and kanamycin and grown overnight at 34° C. shaking at 225 rpm. The confluent culture was added as a 1:20 dilution to LB supplemented with chloramphenicol and kanamycin. To this culture, 20% w/v arabinose was added to a final concentration of 0.2% arabinose to induce the OTS encoded by pAzFRS.2.t1, 100 mM PrOF in 0.2 M NaOH was added to a final concentration of 1 mM PrOF, and 1 N HCl was added to a final concentration of 2 mM. The culture was grown at 34° C. shaking at 225 rpm for 3 h to induce the OTS encoded by pAzFRS.2.t1, after which anhydrotetracycline (aTc) was added to a final concentration of 60 ng/μL to induce pili containing fimA A109PrOF from pSHDS.109. Then, the culture was grown at 34° C. shaking at 135 rpm for an additional 8 h, after which cells were either imaged with TEM to visualize pili production, assayed for pili production using yeast agglutination, or used for pili protein harvesting and subsequent purification.

### Synthesis of Gold Nanoparticle-Pili (AuNP-Pili):

The pili A109PrOF incorporating an NSAA, PrOF, containing an alkyne, were used to produce the AuNP-pili. 5 nm NHS-activated gold nanoparticles (Cytodiagnostics) were reacted with 11-Azido-3,6,9-trioxaundecan-1-amine (Sigma Aldrich) using the recommended protocol from Cytodiagnostics. In the last step, AuNP-linker-azide were obtained by buffer exchanging with PBS pH 7.4 (−) MgCl₂ (−) CaCl₂ using 100 kDa Amicon Ultra centrifugal filter units (three times, centrifuging at 14,000×g for 15 min). Next, AuNP-linker-azide were coupled onto the pili A109PrOF through copper-catalysed azide-alkyne cycloaddition reaction. Briefly, 20 μL of 50 mM THPTA and 10 μL of 20 mM CuSO₄ were premixed for 30 min at room temperature. Later, 10 μL of 150 μM pili were diluted in 30 L of PBS pH 7.4 (−) MgCl₂ (−) CaCl₂ and 2.5 μL of AuNP-linker-azide were added to the mixture. Then, 1 μL of the mixture of THPTA/CuSO4 was added to the entire 40 μL pili/AuNP mixture, are combined. Finally, 2.5 μL of 100 mM aminoguanidine and 2.5 μL of 100 mM sodium ascorbate are added. The reaction mixture was shaken at 500 rpm tabletop shaker (Santa Cruz Biotechnology) for 1 h. To stop the reaction and obtain the AuNP-linker-pili, the samples were buffer exchanged to 150 mM ETA pH 10.5 using 100 kDa Amicon Ultra centrifugal filter units (three times, centrifuging at 14,000×g for 15 min).

### Results

Next, experiments were designed to determine if the generation of a hybrid organic-inorganic pili biomaterial through the site-specific incorporation of nsAAs capable of click chemistry conjugated to AuNPs could further increase conductivity of type 1 pili. Using the GRO and the previously characterized pAzFRS.2.t1 orthogonal translation system (OTS) (Lajoie et al., Science 342, 357-360 (2013), Amiram et al., Nature Biotechnology 33, 1272-1279 (2015)), the incorporation of select nsAAs into pili (Table 4) was tested.

TABLE 4
nsAAs inserted, position, and conductivity

| | | |
|---|---|---|
| Position and nsAA | Pili expression | σ (mS/cm) |

High Aromaticity

| | | |
|---|---|---|
| A80 2NaA (3-(2-Naphthyl)-L-alanine) | Not detected | 2.709 ± 0.1674 |
| A109 2NaA | Yes | |
| A80 PhF (4-phenyl-phenylalanine) | Not detected | |
| A109 PhF | Not detected | |
| A109 4ClF (4-chloro-phenylalanine) | Not detected | |
| A109 4BrF (4-bromo-phenylalanine) | Not detected | |
| A109 4IF (4-bromo-phenylalanine) | Not detected | |
| A109 pAcF | Not detected | |
| (para-acetyl-L-phenylalanine) | | |
| A109 pAF | Not detected | |
| (para-amino-L-phenylalanine) | | |

Conjugation Scaffold

| | | |
|---|---|---|
| A80 PrOF (4-propargyloxy-L- | Not detected | 87.39 ± 8.607 |
| phenylalanine) | | Not Measured |
| A109 PrOF—AuNP | Yes | |
| A80 pAzF (para-azido-L-phenylalanine) | Yes | |
| A109 pAzF | Not detected | |

Previous work reported the nsAA incorporation efficiency of pAzFRS.2.t1 used in this work to be >95% (Amiram et al., Nature Biotechnology 33, 1272-1279 (2015)). nsAA molecules with high aromaticity (3-(2-Naphthyl)-L-alanine: [2NaA]) or those compatible with Cu-catalysed click chemistry (4-azido-L-phenylalanine [pAzF], PrOF) were chosen. Guided by the modified pili containing the aromatic amino acids, mutant pili expression was tested using a yeast agglutination assay (Sheikh et al., PLoS Negl Trop Dis 11, e0005586 (2017), Korhonen, FEMS Microbiology Letters 6, 421-425 (1979)) and it was found that 2NaA and pAzF could be incorporated at FimA position 80 but not position 109, whereas PrOF (FIG. 1D) could be incorporated at FimA position 109 but not 80. Incorporation of one 2NaA residue per FimA monomer increased pili conductivity ˜5-fold to 2.71±0.16 mS/cm (FIG. 4C, 4E), which although a substantial increase, is significantly less than the conductivity of all other mutant type 1 pili incorporating natural aromatic amino acid mutations (FIGS. 2E, 4C, 4E).

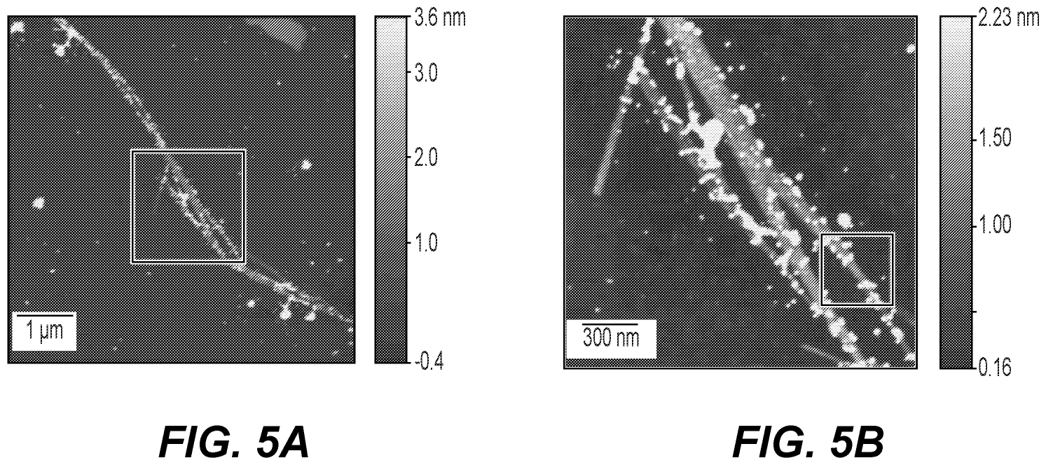

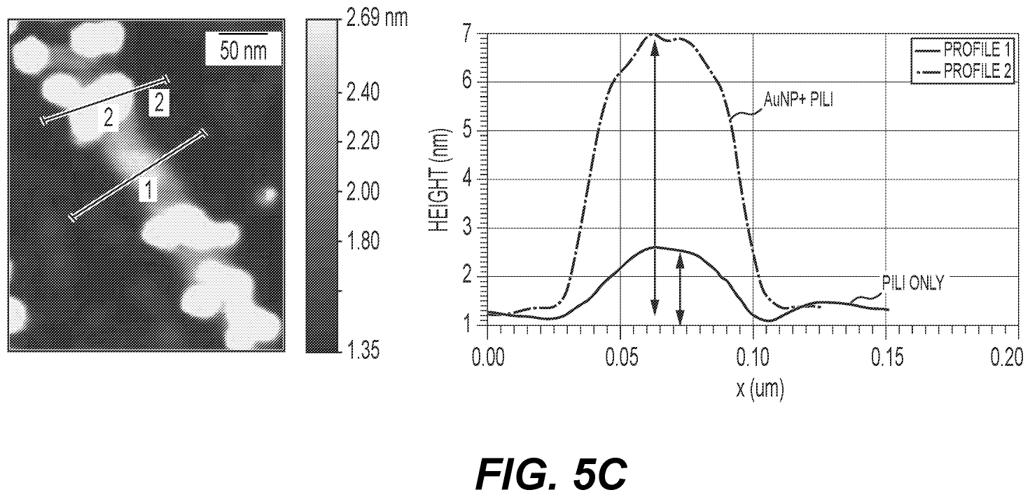

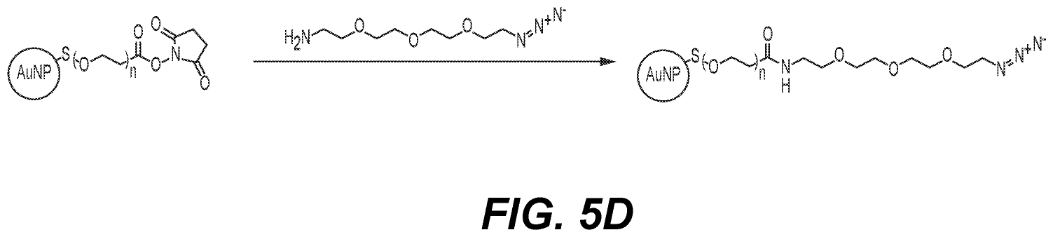

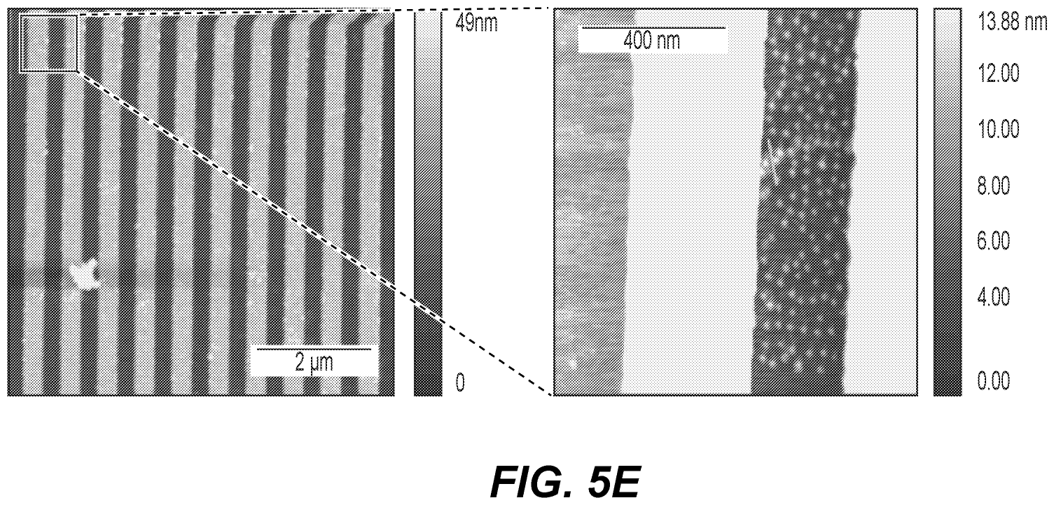

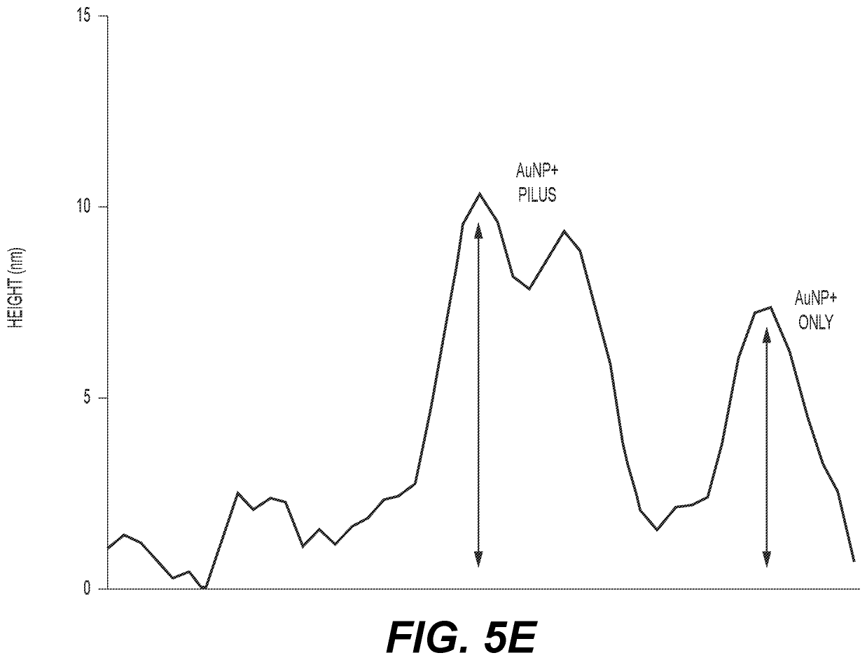

Pili with PrOF inserted at position 109 in every FimA monomer was chosen as a scaffold for site-specific conjugation of AuNP to construct a hybrid organic-inorganic biomaterial. AuNPs, whose surface was covered with terminal azide groups, were conjugated to the terminal alkyne moieties of the PrOF residues (FIG. 1D) through the highly efficient Cu-catalysed click chemistry reaction (Tiwari et al., Chemical Reviews 116, 3086-3240 (2016), Meldal & Tornøe, Chemical Reviews 108, 2952-3015 (2008)) (FIG. 5D). Using AFM imaging it was found that PrOF-pili reacted with azide-AuNPs in the presence of copper were decorated with AuNPs (FIG. 4A, 5D) whereas PrOF-pili reacted with azide-AuNPs without the copper catalyst had no AuNPs attached (FIG. 4B). These results show that this conjugation proceeds through the selective Cu-catalysed click reaction between the incorporated nsAA PrOF residues in the pili and the azide moieties on the AuNPs. The AFM images of the AuNPs conjugated along the pili filament (FIG. 4A, 5A-5E) provide direct visual evidence of efficient and precise conjugation of AuNPs at PrOF residues. Interestingly, the height of pili reacted with copper decreased from 6 nm to 2 nm, potentially due to physical stress from reacting with AuNPs or copper ions altering pili structure (FIG. 4B, FIG. 5C).

Finally, the engineered pili conjugated to AuNPs were placed on gold electrodes separated by 300 nm nonconductive gaps and individual filaments bridging two electrodes were isolated using AFM to measure conductivity of the engineered individual filaments as described above. The AuNP-pili hybrid filaments were found to have conductivity higher than that of all other measured filaments at 87.40±8.91 mS/cm (FIG. 4D, 4E), an increase of ˜170-fold (FIG. 4D-5E). Taken together, the high accuracy of encoding nsAAs (Lajoie et al., Science 342, 357-360 (2013), Amiram et al., Nature Biotechnology 33, 1272-1279 (2015)), the high efficiency of the Cu-catalysed click reaction (Tiwari et al., Chemical Reviews 116, 3086-3240 (2016), Meldal & Tornøe, Chemical Reviews 108, 2952-3015 (2008)), and the AFM images demonstrating the conjugation of AuNP to the PrOF residues demonstrate the construction of hybrid organic-inorganic biomaterials with significantly enhanced conductivity.

These experiments combine the precise, site-specific engineering of bacterial nanowires facilitated using synthetic biology methods with highly accurate conductivity measurements on individual filaments and filament nanostructures to demonstrate the production of multi-functional, highly conductive pili biomaterials. Guided by cryo-EM structures (Hospenthal et al., Structure 25, 1829-1838.e1824 (2017), Spaulding et al., eLife 7, e31662 (2018)), MAGE was used to generate a targeted combinatorial library of genomic type 1 pili mutants to screen for aromatic amino acid mutations that retain pili self-assembly and exhibit increased conductivity. Notably, pili mutants exhibited a wide range of electronic conductivities based on the insertion site and aromaticity of the amino acids inserted into the pili. The insertion of aromatic amino acids into type 1 pili increased pili up to 84-fold with the double tryptophan mutant. Use of single-filament conductivity measurements allowed for the assignment of an increase in conductivity of a filament to the incorporation of aromatic amino acids, providing a higher level of engineered pili characterization compared to commonly used film measurements. The use of molecular dynamics simulations demonstrated more frequent association between pilus proteins in the presence of HMD, which informed the construction of hierarchical multidimensional nanostructures that demonstrated the efficient transport of charges over micrometre distances under ordinary thermal conditions. Bundling of pili increased conductivity 5-fold over the micrometre scale, implying that bundling may further facilitate electron transport down the filaments.

Finally, using the genomically recoded organism (GRO) and a highly efficient orthogonal translation system (OTS) able to incorporate nsAAs into proteins with >95% efficiency (Amiram et al., Nature Biotechnology 33, 1272-1279 (2015)), 2NaA, pAzF, and PrOF were inserted at genetically encoded locations within the FimA monomer and thus across the entire surface of the pilus. Azide-covered AuNPs were then conjugated to the terminal alkyne groups on PrOF residues in pili using the Cu-catalyzed cycloaddition “click” chemistry reaction. Using PrOF to directly label pili proteins with AuNP at single-residue precision is a new strategy presented herein. The use of GROs and an efficient OTS to incorporate “click”-functional nsAAs into a bacterial nanowire allowed production of a new class of hybrid organic-inorganic biomaterials which would otherwise be difficult to produce at scale without recoded bacteria capable of efficiently incorporating nsAAs. The efficient and site-specific incorporation of PrOF into individual FimA monomers allowed pili, large filaments made of polymerized FimA protein, to be uniformly covered in AuNPs—this advance demonstrates the ability to site-specifically functionalize large, micrometre-scale protein structures by re-purposing open codons (e.g., UAG) to encode nsAAs at high efficiency in a recoded bacterial host. This functionalization allowed the pili to be used as a chassis for a hybrid organic-inorganic material engineered with conductivity ˜170-fold higher than wild-type.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A fimbrial polypeptide comprising one or more mutations with an aromatic amino acid relative to the corresponding wildtype fimbrial protein.

2. The fimbrial polypeptide of claim 1, wherein the substitution(s) comprise one or more additions and/or substitutions.

3. The fimbrial polypeptide of claim 1, wherein the aromatic amino acid is selected from phenylalanine, tyrosine, histidine, and tryptophan.

4. The fimbrial polypeptide of claim 1, wherein the aromatic amino acid is a non-standard amino acid.

5. The fimbrial polypeptide of claim 4, wherein the non-standard amino acid is a substrate for a click chemistry reaction.

6. The fimbrial polypeptide of claim 5, wherein the click chemistry reaction comprises copper(I)-catalyzed azide-alkyne cycloaddition (CuAAC), strain-promoted azide-alkyne cycloaddition (SPAAC), strain-promoted alkyne-nitrone cycloaddition (SPANC); or strained alkene reactions optionally selected from alkene-azide [3+2]cycloaddition, the alkene-tetrazine inverse-demand Diels-Alder cycloaddition, and the alkene-tetrazole photoclick reaction.

7. The fimbrial polypeptide of claim 4, wherein the non-standard amino acid(s) is propargyloxy-phenylalanine (PrOF), p-azido-1-phenylalanine (pAzF), 3-(2-Naphthyl)-L-alanine (2NaA), 4-Chloro-phenylalanine, 4-bromo-phenylalanine, para-acetyl-phenylalanine, para-amino-phenylalanine, 4-Iodo-phenylalanine, phenyl-L-phenylalanine, O-2-azidoethyl-tyrosine, para-azidomethyl-phenylalanine, 4-propargyloxy-1-phenylalanine (pPR), pAcF, StyA, 4IF, 4BrF, 4ClF, 4MeF, 4Cf3F, MeY, 4NO2F, 4BuF, BuY, and/or PheF.

8. The fimbrial polypeptide of claim 1, wherein the aromatic amino acid(s) comprises a metal particle, optionally a nanoparticle, or a heme group conjugated thereto.

9. The fimbrial polypeptide of claim 8, wherein the metal is gold.

10. The fimbrial polypeptide of claim 9, wherein the aromatic amino acid is PrOF.

11. The fimbrial polypeptide of claim 1 comprising two or more mutations in close enough proximity to each other to facilitate efficient electron transfer, optionally<15 Å apart, in an assembled pilus formed thereof.

12. The fimbrial polypeptide of claim 1, wherein the wildtype protein is a FimA, optionally E. coli FimA, optionally wherein the E. coli FimA comprises the amino acid sequence of SEQ ID NO:1.

13. The fimbrial polypeptide of claim 1, comprising at least 70% sequence identity to the wildtype protein.

14. The fimbrial polypeptide of claim 12 comprising mutations at one or more of A80, H82, and A109 relative to the wildtype protein.

15. The fimbrial polypeptide of claim 14 comprising phenylalanine, tyrosine, histidine, and/or tryptophan substitutions at one or more of A80, H82, and A109 relative to the wildtype protein, optionally, wherein the mutation(s) comprise or consist of A80F, A109F, A80Y, A80 W, A109 W, A109Y, A80F A109F (double mutant), A80Y A109Y (double mutant), or A80 W A109 W (double mutant).

16. The fimbrial polypeptide of claim 14, comprising propargyloxy-phenylalanine (PrOF), p-azido-1-phenylalanine (pAzF), 3-(2-Naphthyl)-L-alanine (2NaA), 4-Chloro-phenylalanine, 4-bromo-phenylalanine, para-acetyl-phenylalanine, para-amino-phenylalanine, 4-Iodo-phenylalanine, phenyl-L-phenylalanine, O-2-azidoethyl-tyrosine, para-azidomethyl-phenylalanine, 4-propargyloxy-1-phenylalanine (pPR), pAcF, StyA, 4IF, 4BrF, 4ClF, 4MeF, 4Cf3F, MeY, 4NO2F, 4BuF, BuY, and/or PheF substitutions at one or more of A80, H82, and A109 relative to the wildtype protein, optionally, wherein the mutation(s) comprise or consist of PrOF substitution at A109, or pAzF or 2NaA pAzF or 2NaA substitution at A80; optionally wherein the aromatic amino acids comprise a gold nanoparticle conjugate thereto.

17. A pilus comprising a plurality of the fimbrial polypeptide of claim 1.

18. A bundle of pili comprising a plurality of the pilus of claim 17.

19. The bundle of pili of claim 18, wherein the bundle is a 1D, 2D, or 3D bundle.

20. The bundle of pili of claim 18, wherein the bundle is ordered.

21. The bundle of pili of claim 18 comprising a lattice structure.

22. The bundle of pili of claim 18 assembled with an inducer.

23. The bundle of pili of claim 22, wherein the inducer is selected from hexamethylenediamine (HMD), pimelic acid, and 1,3-propanedisulfonic acid.

24. The fimbrial polypeptide of claim 1, or a pilus or bundle of pili formed therefrom, wherein the fimbrial polypeptide, pilus, or bundle of pili is electrically conductive.

25. The fimbrial polypeptide of claim 24, or a pilus or bundle of pili formed therefrom, wherein the fimbrial polypeptide, pilus, or bundle of pili is more conductive than the corresponding wildtype fimbrial polypeptide, pilus, or bundle of pili.

26. An electrical circuit comprising the fimbrial polypeptide of claim 1, or a pilus or bundle of pili formed therefrom.

27. A device comprising the claim 26.

28. The device of claim 27, wherein the device is a sensor, transistor, capacitor, electronic prosthetics, implantable electrode, flexible electronic, energy storage, soft robotics, computing, or information storage.

29. A system comprising the device of claim 28.

30. The electrical circuit claim 26, or a device or system formed therewith, wherein the fimbrial polypeptide, pilus, or bundle of pili serves as the conductive element of the circuit, device, or system.

31. A method of making a fimbrial polypeptide comprising one or more iterations of an aromatic non-standard amino acid comprising expressing a messenger RNA (mRNA) encoding the fimbrial polypeptide in a system comprising:
an orthogonal translation system (OTS) comprising a nucleic acid sequence encoding an aminoacyl tRNA synthetase (AARS) and its cognate tRNA operably linked to expression control sequences and transformed, transfected, or integrated into a genomically recoded organism (GRO) with at least one codon reduced or absent from its genome, and
a plurality of the non-standard amino acid,
wherein the mRNA comprises a nucleic acid sequence comprising at least one iteration of the codon deleted from the GRO,
wherein the AARS can charge the tRNA with non-standard amino acid, and
wherein the tRNA comprises and anticodon that can bind to the codon reduced or absent from the GRO.

32. A method of making pili comprising making the fimbrial polypeptide according to the method of claim 31 in a prokaryotic host, optionally wherein the prokaryotic host is E. coli, and isolating pili formed by the host.

33. A method of forming an ordered bundle of pili comprising isolating pili made according to the method of claim 32, and contacting the pili with an inducer, optionally wherein the inducer is HMD.

34. A method of conducting electricity comprising connecting the pili of claim 32, or a bundle of pili formed therefrom, to two electrodes and applying electricity to one of the electrodes.
